(19) United States
(12) Patent Application Publication
(10) Pub. No.: US 2019/0117705 A1
(43) Pub. Date: Apr. 25, 2019

(54) COMPOSITIONS COMPRISING HUMAN PLACENTAL PERFUSATE CELLS, SUBPOPULATIONS THEREOF, AND THEIR USES

(71) Applicant: CELULARITY, INC., Warren, NJ (US)

(72) Inventors: Jodi P. Gurney, Chicago, IL (US); Xiaokui Zhang, Livingston, NJ (US); Stacy Herb, Mountain Top, PA (US); Robert J. Hariri, Bernardsville, NJ (US)

(73) Assignee: CELULARITY, INC., Warren, NJ (US)

(21) Appl. No.: 16/199,116

(22) Filed: Nov. 23, 2018

Related U.S. Application Data
(63) Continuation of application No. 15/036,649, filed on May 13, 2016, now abandoned.
(60) Provisional application No. 61/905,076, filed on Nov. 15, 2013.
(60) Provisional application No. 61/905,077, filed on Nov. 15, 2013.

(51) Int. Cl. A61K 35/50; A61K 35/28

(52) U.S. Cl. CPC A61K 35/50; A61K 2035/124; A61K 35/28

(57) ABSTRACT

Provided herein are compositions comprising mononuclear cells from human placental perfusate and methods of using such cells, including using the cells together with hematopoietic cells, for example to establish chimerism, reduce the severity or duration of graft versus host disease, treat or ameliorate symptoms of sarcopenia, metabolic disorders and hematologic disorders, such as hematologic malignancies, and treat or ameliorate symptoms of ischemic encephalopathy (e.g., hypoxic ischemic encephalopathy) and other central nervous system injuries.

This application claims benefit of U.S. Provisional Patent Application No. 61/905,076, filed Nov. 15, 2013 and U.S. Provisional Patent Application No. 61/905,077, filed Nov. 15, 2013, the disclosures of which are incorporated by reference herein in their entirety.

### 1. FIELD

Provided herein are compositions comprising mononuclear cells from human placental perfusate and methods of using such cells, including using the cells together with hematopoietic cells, for example to establish chimerism, reduce the severity or duration of graft versus host disease, treat or ameliorate symptoms of sarcopenia, metabolic disorders, and hematologic disorders, such as hematologic malignancies, and treat or ameliorate symptoms of ischemic encephalopathy (e.g., hypoxic ischemic encephalopathy) and other central nervous system injuries.

### 2. BACKGROUND

Placental perfusate comprises a collection of placental cells obtained by passage of a perfusion solution through the placental vasculature, and collection of the perfusion fluid from the vasculature, from the maternal surface of the placenta, or both. Methods of perfusing mammalian placentas are described, e.g., in U.S. Pat. Nos. 7,045,146 and 7,255,879. The population of placental cells obtained by perfusion is heterogeneous, comprising, inter alia, CD34⁺ cells, nucleated cells such as granulocytes, monocytes and macrophages, and tissue culture substrate-adherent placental stem cells.

### 3. SUMMARY

Provided herein are compositions comprising isolated human placental perfusate. In particular embodiments, the human placental perfusate comprises at least 6×10⁵ CD34⁺ cells. In some embodiments, the human placental perfusate further comprises a 2-fold greater number of CD34⁺ cells. In some embodiments, the human placental perfusate further comprises a 10-fold greater number of CD34⁺ cells. In some embodiments, the human placental perfusate further comprises a 50-fold greater number of CD34⁺ cells. In a more specific embodiment, the human placental perfusate comprises substantially pure human placental perfusate CD34⁺ cells.

In other particular embodiments, the human placental perfusate comprises at least 5×10⁵CD34⁺CD45⁻ cells. In some embodiments, the human placental perfusate further comprises a 2-fold greater number of CD34⁺CD45⁻ cells. In some embodiments, the human placental perfusate further comprises a 10-fold greater number of CD34⁺CD45⁻ cells. In some embodiments, the human placental perfusate further comprises a 50-fold greater number of CD34⁺CD45⁻ cells. In a more specific embodiment, the human placental perfusate comprises substantially pure human placental perfusate CD34⁺CD45⁻ cells.

In other particular embodiments, the human placental perfusate comprises at least 6×10⁵CD34⁺CD31⁺ cells. In some embodiments, the human placental perfusate further comprises a 2-fold greater number of CD34⁺CD31⁺ cells. In some embodiments, the human placental perfusate further comprises a 10-fold greater number of CD34⁺CD31⁺ cells. In some embodiments, the human placental perfusate further comprises a 50-fold greater number of CD34⁺CD31⁺ cells. In a more specific embodiment, the human placental perfusate comprises substantially pure human placental perfusate CD34⁺CD31⁺ cells.

In other particular embodiments, the human placental perfusate comprises at least 5×10⁵CD34⁺KDR⁺ cells. In some embodiments, the human placental perfusate further comprises a 2-fold greater number of CD34⁺KDR⁺ cells. In some embodiments, the human placental perfusate further comprises a 10-fold greater number of CD34⁺KDR⁺ cells. In some embodiments, the human placental perfusate further comprises a 50-fold greater number of CD34⁺KDR⁺ cells. In a more specific embodiment, the human placental perfusate comprises substantially pure human placental perfusate CD34⁺KDR⁺ cells.

In other particular embodiments, the human placental perfusate comprises at least 5×10⁵CD34⁺CXCR4⁺ cells. In some embodiments, the human placental perfusate further comprises a 2-fold greater number of CD34⁺ CXCR4⁺ cells. In some embodiments, the human placental perfusate further comprises a 10-fold greater number of CD34⁺CXCR4⁺ cells. In some embodiments, the human placental perfusate further comprises a 50-fold greater number of CD34⁺CXCR4⁺ cells. In a more specific embodiment, the human placental perfusate comprises substantially pure human placental perfusate CD34⁺CXCR4⁺ cells.

In other particular embodiments, the human placental perfusate comprises at least 6×10⁵CD34⁺CD38⁻ cells. In some embodiments, the human placental perfusate further comprises a 2-fold greater number of CD34⁺CD38⁻ cells. In some embodiments, the human placental perfusate further comprises a 10-fold greater number of CD34⁺CD38⁻ cells. In some embodiments, the human placental perfusate further comprises a 50-fold greater number of CD34⁺CD38⁻ cells. In a more specific embodiment, the human placental perfusate comprises substantially pure human placental perfusate CD34⁺CD38⁻ cells.

In other particular embodiments, the human placental perfusate comprises at least 7×10⁵CD34⁺CD117⁻ cells. In some embodiments, the human placental perfusate further comprises a 2-fold greater number of CD34⁺CD117⁻ cells. In some embodiments, the human placental perfusate further comprises a 10-fold greater number of CD34⁺CD117⁻ cells. In some embodiments, the human placental perfusate further comprises a 50-fold greater number of CD34⁺CD117⁻ cells. In a more specific embodiment, the human placental perfusate comprises substantially pure human placental perfusate CD34⁺CD117⁻ cells.

In other particular embodiments, the human placental perfusate comprises at least 6×10⁵CD34⁺CD140a⁺ cells. In some embodiments, the human placental perfusate further comprises a 2-fold greater number of CD34⁺CD140a⁺ cells. In some embodiments, the human placental perfusate further comprises a 10-fold greater number of CD34⁺CD140a⁺ cells. In some embodiments, the human placental perfusate further comprises a 50-fold greater number of CD34⁺CD140a⁺ cells. In a more specific embodiment, the human placental perfusate comprises substantially pure human placental perfusate CD34⁺CD140a⁺ cells.

In other particular embodiments, the human placental perfusate comprises at least 3×10⁵CD34⁺Nestin⁺ cells. In some embodiments, the human placental perfusate further comprises a 2-fold greater number of CD34⁺Nestin⁺ cells. In some embodiments, the human placental perfusate further comprises a 10-fold greater number of CD34⁺Nestin⁺ cells. In some embodiments, the human placental perfusate further comprises a 50-fold greater number of CD34⁺Nestin⁺ cells. In a more specific embodiment, the human placental perfusate comprises substantially pure human placental perfusate CD34⁺Nestin⁺ cells.

In other particular embodiments, the human placental perfusate comprises at least 3×10⁴CD3⁺CD4⁺CD8⁻CD25^{hi}CD127^{low} cells. In some embodiments, the human placental perfusate further comprises a 2-fold greater number of CD3⁺CD4⁺CD8⁻CD25^{hi}CD127^{low} cells. In some embodiments, the human placental perfusate further comprises a 10-fold greater number of CD3⁺CD4⁺CD8⁻CD25^{hi}CD127^{low} cells. In some embodiments, the human placental perfusate further comprises a 50-fold greater number of CD3⁺CD4⁺CD8⁻CD25^{hi}CD127^{low} cells. In a more specific embodiment, the human placental perfusate comprises substantially pure human placental perfusate CD3⁺CD4⁺CD8⁻CD25^{hi}CD127^{low} cells.

In some embodiments, the human placental perfusate has been isolated from perfusion of a single placenta.

Also provided herein are methods of treating a central nervous system injury, disease, or disorder in a subject, comprising administering to the subject a composition comprising isolated human placental perfusate provided herein and hematopoietic cells from another source. In a particular embodiment, said central nervous system injury, disease, or disorder is ischemic encephalopathy (e.g., hypoxic ischemic encephalopathy).

Also provided herein are methods of treating sarcopenia in a subject, comprising administering to the subject a composition comprising isolated human placental perfusate provided herein and hematopoietic cells from another source.

Provided herein are methods of inducing chimerism in a subject, comprising administering to the subject a composition comprising isolated human placental perfusate provided herein and hematopoietic cells from another source.

Provided herein are methods for cell engraftment in a subject, comprising administering to the subject a composition comprising isolated human placental perfusate provided herein and hematopoietic cells from another source.

Provided herein are methods for reducing the duration or severity of graft versus host disease (GVHD) in a subject, comprising administering to the subject a composition comprising isolated human placental perfusate provided herein and hematopoietic cells from another source.

Provided herein are methods of treating a metabolic disorder in a subject, comprising administering to the subject a composition comprising isolated human placental perfusate provided herein and hematopoietic cells from another source.

Provided herein are methods of treating a hematologic disorder or malignancy in a subject, comprising administering to the subject a composition comprising isolated human placental perfusate provided herein and hematopoietic cells from another source.

Provided herein are compositions comprising isolated human placental perfusate or human placental perfusate cells for use in a method (a) of treatment of a central nervous system injury, disease, or disorder in a subject, preferably said central nervous system injury, disease, or disorder is hypoxic ischemic encephalopathy; (b) of inducing chimerism in a subject; (c) for cell engraftment; (d) for reducing the duration or severity of graft versus host disease (GVHD) in a subject; (e) of treating a metabolic disorder in a subject; (f) of treating a hematologic disorder or malignancy in a subject; or (g) of treating sarcopenia in a subject.

Also provided herein are compositions comprising isolated human placental perfusate or human placental perfusate cells for use in a method (a) of treatment of a central nervous system injury, disease, or disorder in a subject, preferably said central nervous system injury, disease, or disorder is hypoxic ischemic encephalopathy; (b) of inducing chimerism in a subject; (c) for cell engraftment; (d) for reducing the duration or severity of graft versus host disease (GVHD) in a subject; (e) of treating a metabolic disorder in a subject; (f) of treating a hematologic disorder or malignancy in a subject; or (g) of treating sarcopenia in a subject, wherein the composition further comprises hematopoietic cells from another source.

### 4. BRIEF DESCRIPTION OF THE DRAWINGS

FIG. 1 depicts the total nucleated cell count for forty-three matched pairs of human placental perfusate and umbilical cord blood units.

FIGS. 2A-2C depict the FACS analysis of human placental perfusate cells (A) gated first for CD45⁺ cells (B) and gated first for CD34⁺ cells (C).

FIGS. 3A-3E depict a comparison between human placental perfusate (A) and umbilical cord blood (B) gated first for CD34⁺ cells. The human placental perfusate cells gated for CD34⁺ cells (C) may then be sorted to separate CD34⁺CD45⁻ (D) and CD34⁺CD45⁺ (E) cells.

FIG. 4 depicts the percentage of nucleated cells expressing specific CD34⁺ phenotypes in human placental perfusate (HPP) or cord blood (HUCB).

FIG. 5 depicts a lipoprotein uptake experiment using human placental perfusate endothelial cells (upper) and micro-vessel formation observed in HUVECs and human placental perfusate (HPP) cells (lower).

FIG. 6 depicts the percentage of nucleated cells expressing CD34 and/or Nestin in human placental perfusate (HPP) or cord blood (HUCB).

FIG. 7 depicts the percentage of nucleated cells expressing specific HLA antigens in human placental perfusate (HPP) or cord blood (HUCB).

FIG. 8 depicts the percentage of nucleated cells expressing CD3 with or without CD4 and with or without CD8 in human placental perfusate or cord blood (HUCB).

### 5. DETAILED DESCRIPTION

In various aspects, provided herein are methods of producing mononuclear cells from human placental perfusate (HPCs), e.g. human placental perfusate, compositions comprising such cells, and the use of such cells in the treatment of individuals having a central nervous system injury, disease, disorder or condition. In a more specific embodiment, said disease, disorder or condition is ischemic encephalopathy (e.g., hypoxic ischemic encephalopathy). Also provided herein are methods of administering HPCs, e.g. human placental perfusate, to a subject, e.g. a human subject, to reduce the severity of graft versus host disease and to treat or ameliorate symptoms of metabolic and hematologic disorders, such as hematologic malignancies. Also provided herein are methods of administering HPCs, e.g. human placental perfusate, to a subject, e.g. a human subject, to treat or ameliorate symptoms of sarcopenia.

5.1 Compositions Comprising Placental Perfusate Cells and Methods of Using them

Placental perfusate comprises total mononuclear cells obtained from perfusion solution that has passed through the placenta, as described herein. Typically, placental perfusate from a single placental perfusion comprises about 100 million to about 500 million nucleated cells. In certain embodiments, placental perfusate from a single placental perfusion comprises about 100 million to about 400 million nucleated cells, about 100 million to about 300 million nucleated cells, or about 100 million to about 200 million nucleated cells.

Mononuclear cells from human placental perfusate (HPCs), e.g., human placental perfusate, for use in accordance with the present disclosure may be collected in any medically or pharmaceutically-acceptable manner and may be present in a composition, e.g., a pharmaceutical composition. In certain embodiments, a composition (e.g., a pharmaceutical composition, i.e., a pharmaceutical grade solution suitable for administration to a human) provided herein comprises human placental perfusate.

In certain embodiments, the placental perfusate or perfusate cells comprise CD34⁺ cells, e.g., hematopoietic stem or progenitor cells or endothelial progenitor cells. Such cells can, in a more specific embodiment, comprise CD34⁺CD45⁻ stem or progenitor cells, CD34⁺CD45⁺ stem or progenitor cells, myeloid progenitors, lymphoid progenitors, and/or erythroid progenitors.

In other embodiments, the placental perfusate and placental perfusate cells comprise, e.g., endothelial progenitor cells, osteoprogenitor cells, and/or natural killer cells.

In certain embodiments, placental perfusate as collected from the placenta and depleted of erythrocytes, or perfusate cells isolated from such perfusate, comprise about 60-90%, e.g., about 60%, 65%, 70%, 80%, 85%, or 90%, for example, about 60-90%. 65-90%, 70-90% or about 75-90% leukocytes. In certain embodiments, placental perfusate as collected from the placenta and depleted of erythrocytes, or perfusate cells isolated from such perfusate, comprise about 2-11%, e.g., about 2, 3, 4, 5, 6, 7, 8, 9, 10 or 11%, for example, about 5-8%, or about 6-7% natural killer cells (CD3⁻, CD56⁺); and/or about 7-37%, e.g., about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37%, for example, about 20-25%, about 22-24%, or about 22-23% T cells (CD3⁺); and/or about 5-15%, e.g., about 5, 6, 7, 8, 9, 10, 11, 12, 13, 14, or 15%, for example, about 8-12%, or about 10-11% B cells (CD19⁺); and/or about 20-32%, e.g., about 20, 21, 22, 23, 24, 25, 26, 27, 28, 29, 30, 31, or 32%, e.g., about 22-28%, 25-28%, or about 26-27% monocytes (CD14⁺); and/or about 1-5%, e.g., about 1, 2, 3, 4, or 5%, for example about 2-4% or about 2-3% endothelial progenitor cells (e.g., CD34⁺, CD31⁺); and/or about 0.5-5%, e.g., about 0.5, 1, 2, 3, 4, or 5%, for example about 2-4% or about 2-3% neural progenitor cells (Nestin⁺); and/or about 1-7%, e.g., about 1, 2, 3, 4, 5, 6, or 7%, for example about 2-4% or about 3-4% hematopoietic progenitor cells (CD34⁺); and/or about 1-5%, e.g., about 1, 2, 3, 4, or 5%, for example about 2-4%, about 2-3%, or about 1-2% adherent placental stem cells (e.g., CD34⁻, CD117⁻, CD105⁺ and CD44⁺), as determined, e.g. by flow cytometry, e.g., by FACS analysis.

In certain embodiments, said placental perfusate cells comprise CD34⁺ cells. In a more specific embodiment, said CD34⁺ cells are CD34⁺CD45⁻ cells. In another embodiment, said CD34⁺ cells are isolated from placenta. In yet another embodiment, said population of placental perfusate cells further comprises additional isolated CD34⁺ cells not isolated from said perfusate (e.g., isolated from umbilical cord blood, placental blood, peripheral blood, bone marrow, or the like). In another embodiment, said additional CD34⁺ cells are isolated from umbilical cord blood, placental blood, peripheral blood, or bone marrow.

In other embodiments, the CD34⁺ cells are additionally CD117⁻. In certain embodiments, the CD34⁺ cells are additionally CD31⁺, CXCR4⁺, and/or KDR⁺. In certain embodiments, the CD34⁺ cells are additionally CD140a⁺. In certain embodiments, the CD34⁺ cells are additionally Nestin⁺. In certain embodiments, said human placental perfusate cells, e.g. said CD34⁺ cells, comprise more CD117⁻ cells than the equivalent number of cells from umbilical cord blood. In certain embodiments, said CD34⁺ cells comprise more CD31⁺, CXCR4⁺, and/or KDR⁺ cells than the equivalent number of cells from umbilical cord blood. In certain embodiments, any of said CD34⁺ cells are CD34⁺CD45⁻ cells. In certain embodiments, said human placental perfusate cells, e.g. said CD34⁺ cells, comprise more CD140a⁺ cells than the equivalent number of cells from umbilical cord blood. In certain embodiments, said human placental perfusate cells, e.g. said CD34⁺ cells, comprise more Nestin⁺ cells than the equivalent number of cells from umbilical cord blood.

In another specific embodiment, said placental perfusate cells, e.g., said CD34⁺ cells produce amounts of one or more angiogenesis-related markers at a higher level than an equivalent number of CD34⁺ cells from umbilical cord blood. In specific embodiments, said markers comprise CD31, KDR and/or CXCR4. In a particular embodiment, said CD34⁺ cells are CD45⁻. In a more specific embodiment, said CD34⁺ cells or CD34⁺CD45⁻ cells express a higher level of at least one of CD31, CXCR4 or KDR than an equivalent number of CD34⁺ cells from umbilical cord blood. In certain embodiments, said placental perfusate cells, e.g., said CD34⁺ placental cells express a higher level of Nestin than the equivalent number of cells from umbilical cord blood.

In another specific embodiment, said placental perfusate is enriched for CD34⁺ cells. In certain embodiments, said placental perfusate is enriched for CD45⁻ cells. In certain embodiments, said placental perfusate is enriched for CD34⁺CD45⁻ cells. In certain embodiments, said placental perfusate is enriched for CD31⁺, KDR⁺ and/or CXCR4⁺ cells. In certain embodiments, said placental perfusate is enriched for CD34⁺CD31⁺, CD34⁺KDR⁺, and/or CD34⁺CXCR4⁺ cells. In certain embodiments, said placental perfusate is enriched for CD140a⁺ cells. In certain embodiments, said placental perfusate is enriched for CD34⁺CD140a⁺ cells. In certain embodiments, said placental perfusate is enriched for CD117⁻ cells. In certain embodiments, said placental perfusate is enriched for CD34⁺CD117⁻ cells. In certain embodiments, said placental perfusate is enriched for CD38⁻ cells. In certain embodiments, said placental perfusate is enriched for CD34⁺CD38⁻ cells. In certain embodiments, said placental perfusate is enriched for Nestin⁺ cells. In certain embodiments, said placental perfusate is enriched for CD34⁺Nestin⁺ cells. In certain embodiments, said placental perfusate is enriched for CD3⁺CD4⁺CD8⁻CD25^{hi}CD127^{low} cells.

With respect to enrichment, a particular cell population can be enriched for one or more cell types, e.g., cells exhibiting a specific cell surface marker phenotype, by, for example, introducing such cell type(s) into the population, adding additional amounts of the cell type(s) into the population, and/or depleting (removing some or all of) one or more different cell types, e.g., cells exhibiting a different specific cell surface marker phenotype, from the population.

In some embodiments, enrichment of a particular population or subpopulation of cells in said placental perfusate or placental perfusate cells is accomplished via one or more rounds of cell sorting, e.g., FACS cell sorting. In some embodiments, enrichment of a particular population or subpopulation of cells in said placental perfusate or placental perfusate cells is accomplished via removal of one or more other populations or subpopulations of cells. In some embodiments, enrichment of a particular population or subpopulation of cells in said placental perfusate or placental perfusate cells is accomplished via addition of a population or subpopulation of cells that have been isolated from placental perfusate. In some embodiments, enrichment of a particular population or subpopulation of cells in said placental perfusate or placental perfusate cells is accomplished via addition of a population or subpopulation of cells that have been isolated from another source (e.g. umbilical cord blood). In some embodiments, enrichment of a particular population or subpopulation of cells in said placental perfusate or placental perfusate cells is accomplished via addition of placental perfusate that has been enriched for that population or subpopulation of cells. In other embodiments, enrichment of a particular population or subpopulation of cells in said placental perfusate or placental perfusate cells is accomplished via expansion of that population or subpopulation of cells. In some embodiments, enrichment of a particular population or subpopulation of cells in said placental perfusate or placental perfusate cells is accomplished by increasing the total number of those cells in said placental perfusate or placental perfusate cells. In some embodiments, enrichment of a particular population or subpopulation of cells in said placental perfusate or placental perfusate cells is accomplished by increasing the proportion of those cells in said placental perfusate or placental perfusate cells. In some embodiments, enrichment of a particular population or subpopulation of cells in said placental perfusate or placental perfusate cells is accomplished by expansion of a particular population or subpopulation of cells via culturing. In some embodiments, depletion of a particular population or subpopulation of cells in said placental perfusate or placental perfusate cells is accomplished by expansion of another particular population or subpopulation of cells via culturing. Enrichment for or isolation of a particular population or subpopulation of cells may be performed after expansion of a particular population or subpopulation of cells or may be performed on the total nucleated cells from placental perfusate.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺ cells. In another embodiment, said placental perfusate or said placental perfusate cells comprise 6×10⁵ to 3×10⁷ CD34⁺ cells. In another embodiment, said placental perfusate or said placental perfusate cells comprise 1×10⁶ to 1×10⁷ CD34⁺ cells. In another embodiment, said placental perfusate or said placental perfusate cells comprise 1×10⁵ to 1×10⁸ CD34⁺ cells. In another embodiment, said placental perfusate or said placental perfusate cells comprise 1×10⁴ to 1×10⁸ CD34⁺ cells. In a specific embodiment, said CD34⁺ cells have been obtained through cell sorting of the total nucleated cells from placental perfusate with an antibody against CD34. In some embodiments, said CD34⁺ cells have been isolated from placental perfusate or said placental perfusate cells. In some embodiments, CD34⁺ cells from placental perfusate have been expanded in culture.

In another specific embodiment, said placental perfusate or said placental perfusate cells comprise about 10% CD34⁺ cells. In another embodiment, said placental perfusate or said placental perfusate cells comprise 8% to 12% CD34⁺ cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺CD45⁻ cells. In another embodiment, said placental perfusate or said placental perfusate cells comprise 5×10⁵ to 1×10⁷ CD34⁺CD45⁻ cells. In another embodiment, said placental perfusate or said placental perfusate cells comprise 1×10⁶ to 1×10⁷ CD34⁺CD45⁻ cells. In another embodiment, said placental perfusate or said placental perfusate cells comprise 1×10⁵ to 1×10⁸ CD34⁺CD45⁻ cells. In another embodiment, said placental perfusate or said placental perfusate cells comprise 1×10⁴ to 1×10⁸ CD34⁺CD45⁻ cells. In a specific embodiment, said CD34⁺CD45⁻ cells have been obtained through cell sorting of the total nucleated cells from placental perfusate with an antibody against CD34, followed by sorting of the total nucleated cells from placental perfusate with an antibody against CD45. In another specific embodiment, said CD34⁺CD45⁻ cells have been obtained through cell sorting of the total nucleated cells from placental perfusate with an antibody against CD45, followed by sorting of the total nucleated cells from placental perfusate with an antibody against CD34. In some embodiments, said CD34⁺CD45⁻ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺CD31⁺ cells. In another embodiment, said placental perfusate or said placental perfusate cells comprise 6×10⁵ to 3×10⁷ CD34⁺CD31⁺ cells. In another embodiment, said placental perfusate or said placental perfusate cells comprise 1×10⁶ to 1×10⁷ CD34⁺CD31⁺ cells. In another embodiment, said placental perfusate or said placental perfusate cells comprise 1×10⁵ to 1×10⁸ CD34⁺CD31⁺ cells. In another embodiment, said placental perfusate or said placental perfusate cells comprise 1×10⁴ to 1×10⁸ CD34⁺CD31⁺ cells. In a specific embodiment, said CD34⁺CD31⁺ cells have been obtained through cell sorting of the total nucleated cells from placental perfusate with an antibody against CD34, followed by sorting of the total nucleated cells from placental perfusate with an antibody against CD31. In another specific embodiment, said CD34⁺CD31⁺ cells have been obtained through cell sorting of the total nucleated cells from placental perfusate with an antibody against CD31, followed by sorting of the total nucleated cells from placental perfusate with an antibody against CD34. In some embodiments, said CD34⁺CD31⁺ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺KDR⁺ cells. In another embodiment, said placental perfusate or said placental perfusate cells comprise 5×10⁵ to 2×10⁷ CD34⁺KDR⁺ cells. In another embodiment, said placental perfusate or said placental perfusate cells comprise 1×10⁶ to 1×10⁷ CD34⁺KDR⁺ cells. In another embodiment, said placental perfusate or said placental perfusate cells comprise 1×10⁵ to 1×10⁸ CD34⁺KDR⁺ cells. In another embodiment, said placental perfusate or said placental perfusate cells comprise 1×10⁴ to 1×10⁸ CD34⁺KDR⁺ cells. In a specific embodiment, said CD34⁺KDR⁺ cells have been obtained through cell sorting of the total nucleated cells from placental perfusate with an antibody against CD34, followed by sorting of the total nucleated cells from placental perfusate with an antibody against KDR. In another specific embodiment, said CD34⁺KDR⁺ cells have been obtained through cell sorting of the total nucleated cells from placental perfusate with an antibody against KDR, followed by sorting of the total nucleated cells from placental perfusate with an antibody against CD34. In some embodiments, said CD34⁺KDR⁺ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺ CXCR4⁺ cells. In another embodiment, said placental perfusate or said placental perfusate cells comprise 6×10⁵ to 3×10⁷ CD34⁺ CXCR4⁺ cells. In another embodiment, said placental perfusate or said placental perfusate cells comprise 1×10⁶ to 1×10⁷ CD34⁺ CXCR4⁺ cells. In another embodiment, said placental perfusate or said placental perfusate cells comprise 1×10⁵ to 1×10⁸ CD34⁺ CXCR4⁺ cells. In another embodiment, said placental perfusate or said placental perfusate cells comprise 1×10⁴ to 1×10⁸ CD34⁺ CXCR4⁺ cells. In a specific embodiment, said CD34⁺ CXCR4⁺ cells have been obtained through cell sorting of the total nucleated cells from placental perfusate with an antibody against CD34, followed by sorting of the total nucleated cells from placental perfusate with an antibody against CXCR4. In another specific embodiment, said CD34⁺ CXCR4⁺ cells have been obtained through cell sorting of the total nucleated cells from placental perfusate with an antibody against CXCR4, followed by sorting of the total nucleated cells from placental perfusate with an antibody against CD34. In some embodiments, said CD34⁺ CXCR4⁺ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34CD38⁻ cells. In another embodiment, said placental perfusate or said placental perfusate cells comprise 6×10⁵ to 3×10⁷ CD34CD38⁻ cells. In another embodiment, said placental perfusate or said placental perfusate cells comprise 1×10⁶ to 1×10⁷ CD34CD38⁻ cells. In another embodiment, said placental perfusate or said placental perfusate cells comprise 1×10⁵ to 1×10⁸ CD34CD38⁻ cells. In another embodiment, said placental perfusate or said placental perfusate cells comprise 1×10⁴ to 1×10⁸ CD34CD38⁻ cells. In a specific embodiment, said CD34CD38⁻ cells have been obtained through cell sorting of the total nucleated cells from placental perfusate with an antibody against CD34, followed by sorting of the total nucleated cells from placental perfusate with an antibody against CD38. In another specific embodiment, said CD34CD38⁻ cells have been obtained through cell sorting of the total nucleated cells from placental perfusate with an antibody against CD38, followed by sorting of the total nucleated cells from placental perfusate with an antibody against CD34. In some embodiments, said CD34CD38⁻ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺CD117⁻ cells. In another embodiment, said placental perfusate or said placental perfusate cells comprise 7×10⁵ to 2×10⁷ CD34⁺CD117⁻ cells. In another embodiment, said placental perfusate or said placental perfusate cells comprise 1×10⁶ to 1×10⁷ CD34CD117⁻ cells. In another embodiment, said placental perfusate or said placental perfusate cells comprise 1×10⁵ to 1×10⁸ CD34⁺CD117⁻ cells. In another embodiment, said placental perfusate or said placental perfusate cells comprise 1×10⁴ to 1×10⁸ CD34⁺CD117⁻ cells. In a specific embodiment, said CD34⁺CD117⁻ cells have been obtained through cell sorting of the total nucleated cells from placental perfusate with an antibody against CD34, followed by sorting of the total nucleated cells from placental perfusate with an antibody against CD117. In another specific embodiment, said CD34⁺CD117⁻ cells have been obtained through cell sorting of the total nucleated cells from placental perfusate with an antibody against CD117, followed by sorting of the total nucleated cells from placental perfusate with an antibody against CD34. In some embodiments, said CD34⁺CD117⁻ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺CD140a⁺ cells. In another embodiment, said placental perfusate or said placental perfusate cells comprise 6×10⁵ to 2×10⁷ CD34⁺CD140a⁺ cells. In another embodiment, said placental perfusate or said placental perfusate cells comprise 1×10⁶ to 1×10⁷ CD34⁺CD140a⁺ cells. In another embodiment, said placental perfusate or said placental perfusate cells comprise 1×10⁵ to 1×10⁸ CD34⁺CD140a⁺ cells. In another embodiment, said placental perfusate or said placental perfusate cells comprise 1×10⁴ to 1×10⁸ CD34⁺CD140a⁺ cells. In a specific embodiment, said CD34⁺CD140a⁺ cells have been obtained through cell sorting of the total nucleated cells from placental perfusate with an antibody against CD34, followed by sorting of the total nucleated cells from placental perfusate with an antibody against CD140a. In another specific embodiment, said CD34⁺CD140a⁺ cells have been obtained through cell sorting of the total nucleated cells from placental perfusate with an antibody against CD140a, followed by sorting of the total nucleated cells from placental perfusate with an antibody against CD34. In some embodiments, said CD34⁺CD140a⁺ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD34⁺Nestin⁺ cells. In another embodiment, said placental perfusate or said placental perfusate cells comprise 6×10⁵ to 2×10⁷ CD34⁺Nestin⁺ cells. In another embodiment, said placental perfusate or said placental perfusate cells comprise 1×10⁶ to 1×10⁷ CD34⁺Nestin⁺ cells. In another embodiment, said placental perfusate or said placental perfusate cells comprise 1×10⁵ to 1×10⁸ CD34⁺Nestin⁺ cells. In another embodiment, said placental perfusate or said placental perfusate cells comprise 1×10⁴ to 1×10⁸ CD34⁺Nestin⁺ cells. In a specific embodiment, said CD34⁺Nestin⁺ cells have been obtained through cell sorting of the total nucleated cells from placental perfusate with an antibody against CD34, followed by sorting of the total nucleated cells from placental perfusate with an antibody against Nestin. In another specific embodiment, said CD34⁺Nestin⁺ cells have been obtained through cell sorting of the total nucleated cells from placental perfusate with an antibody against Nestin, followed by sorting of the total nucleated cells from placental perfusate with an antibody against CD34. In some embodiments, said CD34⁺Nestin⁺ cells have been isolated from placental perfusate or said placental perfusate cells.

In another specific embodiment, said placental perfusate or said placental perfusate cells comprise about 2×10⁶, 3×10⁶, 4×10⁶, 5×10⁶, 6×10⁶, 7×10⁶, 8×10⁶, or 9×10⁶ CD3⁺CD4⁺CD8⁻CD25^{hi}CD127^{low} cells. In another embodiment, said placental perfusate or said placental perfusate cells comprise 4×10⁴ to 5×10⁶ CD3⁺CD4⁺CD8⁻CD25^{hi}CD127^{low} cells. In another embodiment, said placental perfusate or said placental perfusate cells comprise 1×10⁶ to 1×10⁷CD3⁺CD4⁺CD8⁻CD25^{hi}CD127^{low} cells. In another embodiment, said placental perfusate or said placental perfusate cells comprise 1×10⁵ to 1×10⁸ CD3⁺CD4⁺CD8⁻ CD25^{hi}CD127^{low} cells. In another embodiment, said placental perfusate or said placental perfusate cells comprise 1×10⁴ to 1×10⁸ CD3⁺CD4⁺CD8⁻CD25^{hi}CD127^{low} cells. In a specific embodiment, said CD3⁺CD4⁺CD8⁻CD25^{hi}CD127^{low} cells have been isolated. In a more specific embodiment, said CD3⁺CD4⁺CD8⁻CD25^{hi}CD127^{low} cells have been isolated using a complete kit for human CD4⁺CD25^{hi}CD127^{low} regulatory T cells (Cat#15861, StemCell).

In certain embodiments, the enrichment in CD34⁺ cells is 2-fold over placental perfusate or placental perfusate cells that have not been enriched. In certain embodiments, the enrichment in CD34⁺ cells is 3-fold over placental perfusate or placental perfusate cells that have not been enriched. In certain embodiments, the enrichment in CD34⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺ cells.

In certain embodiments, the enrichment in CD45⁻ cells is 2-fold over placental perfusate or placental perfusate cells that have not been enriched. In certain embodiments, the enrichment in CD45⁻ cells is 3-fold over placental perfusate or placental perfusate cells that have not been enriched. In certain embodiments, the enrichment in CD45⁻ cells is 2, 3, 4, 5, 6, 7, 8, 9, 10, 15, or 20-fold over placental perfusate or placental perfusate cells that have not been enriched. In another embodiment, the placental perfusate cells are a pure or substantially pure population of CD45⁻ cells.

In certain embodiments, the enrichment in CD34⁺CD45⁻ cells is 2-fold over placental perfusate or placental perfusate cells that have not been enriched. In certain embodiments, the enrichment in CD34⁺CD45⁻ cells is 3-fold over placental perfusate or placental perfusate cells that have not been enriched. In certain embodiments, the enrichment in CD34⁺CD45⁻ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺CD45⁻ cells.

In certain embodiments, the enrichment in CD31⁺, KDR⁺ and/or CXCR4⁺ cells is 2-fold over placental perfusate or placental perfusate cells that have not been enriched. In certain embodiments, the enrichment in CD31⁺, KDR⁺ and/or CXCR4⁺ cells is 3-fold over placental perfusate or placental perfusate cells that have not been enriched. In certain embodiments, the enrichment in CD31⁺, KDR⁺ and/or CXCR4⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD31⁺, KDR⁺ and/or CXCR4⁺ cells.

In certain embodiments, the enrichment in CD34⁺CD31⁺, CD34⁺KDR⁺ and/or CD34⁺ CXCR4⁺ cells is 2-fold over placental perfusate or placental perfusate cells that have not been enriched. In certain embodiments, the enrichment in CD34⁺CD31⁺, CD34⁺KDR⁺ and/or CD34⁺ CXCR4⁺ cells is 3-fold over placental perfusate or placental perfusate cells that have not been enriched. In certain embodiments, the enrichment in CD34⁺CD31⁺, CD34⁺KDR⁺ and/or CD34⁺CXCR4⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺CD31⁺, CD34⁺KDR⁺ and/or CD34 CXCR4⁺ cells.

In certain embodiments, the enrichment in CD117⁻ cells is 2-fold over placental perfusate or placental perfusate cells that have not been enriched. In certain embodiments, the enrichment in CD117⁻ cells is 3-fold over placental perfusate or placental perfusate cells that have not been enriched. In certain embodiments, the enrichment in CD117⁻ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD117⁻ cells.

In certain embodiments, the enrichment in CD34⁺CD117⁻ cells is 2-fold over placental perfusate or placental perfusate cells that have not been enriched. In certain embodiments, the enrichment in CD34⁺CD117⁻ cells is 3-fold over placental perfusate or placental perfusate cells that have not been enriched. In certain embodiments, the enrichment in CD34⁺CD117⁻ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺CD117⁻ cells.

In certain embodiments, the enrichment in CD38⁻ cells is 2-fold over placental perfusate or placental perfusate cells that have not been enriched. In certain embodiments, the enrichment in CD38⁻ cells is 3-fold over placental perfusate or placental perfusate cells that have not been enriched. In certain embodiments, the enrichment in CD38⁻ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD38⁻ cells.

In certain embodiments, the enrichment in CD34⁺CD38⁻ cells is 2-fold over placental perfusate or placental perfusate cells that have not been enriched. In certain embodiments, the enrichment in CD34⁺CD38⁻ cells is 3-fold over placental perfusate or placental perfusate cells that have not been enriched. In certain embodiments, the enrichment in CD34⁺CD38⁻ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺CD38⁻ cells.

In certain embodiments, the enrichment in CD140a⁺ cells is 2-fold over placental perfusate or placental perfusate cells that have not been enriched. In certain embodiments, the enrichment in CD140a⁺ cells is 3-fold over placental perfusate or placental perfusate cells that have not been enriched. In certain embodiments, the enrichment in CD140a⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD140a⁺ cells.

In certain embodiments, the enrichment in CD34⁺CD140a⁺ cells is 2-fold over placental perfusate or placental perfusate cells that have not been enriched. In certain embodiments, the enrichment in CD34⁺CD140a⁺ cells is 3-fold over placental perfusate or placental perfusate cells that have not been enriched. In certain embodiments, the enrichment in CD34⁺CD140a⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺CD140a⁺ cells.

In certain embodiments, the enrichment in Nestin⁺ cells is 2-fold over placental perfusate or placental perfusate cells that have not been enriched. In certain embodiments, the enrichment in Nestin⁺ cells is 3-fold over placental perfusate or placental perfusate cells that have not been enriched. In certain embodiments, the enrichment in Nestin⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of Nestin⁺ cells.

In certain embodiments, the enrichment in CD34⁺Nestin⁺ cells is 2-fold over placental perfusate or placental perfusate cells that have not been enriched. In certain embodiments, the enrichment in CD34⁺Nestin⁺ cells is 3-fold over placental perfusate or placental perfusate cells that have not been enriched. In certain embodiments, the enrichment in CD34⁺Nestin⁺ cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD34⁺Nestin⁺ cells.

In certain embodiments, the enrichment in CD3⁺CD4⁺CD8⁻CD25^{hi}CD127^{low} cells is 2-fold over placental perfusate or placental perfusate cells that have not been enriched. In certain embodiments, the enrichment in CD3⁺CD4⁺CD8⁻CD25^{hi}CD127^{low} cells is 3-fold over placental perfusate or placental perfusate cells that have not been enriched. In certain embodiments, the enrichment in CD3⁺CD4⁺CD8⁻CD25^{hi}CD127^{low} cells is 2, 3, 4, 5, 6, 7, 8, 9, 10, 15, 20, 25, 30, 40 or 50-fold over placental perfusate or placental perfusate cells that have not been enriched. In another embodiment, the placental perfusate cells are a pure or substantially pure population of CD3⁺CD4⁺CD8⁻CD25^{hi}CD127^{low} cells.

In certain embodiments, said placental perfusate cells, e.g., said CD34⁺ cells, express CD3 at a lower level than the equivalent number of cells from umbilical cord blood. In certain embodiments, said placental perfusate cells, e.g., said CD34⁺ cells, express CD3 and CD8 at a lower level than the equivalent number of cells from umbilical cord blood. In certain embodiments, said placental perfusate cells, e.g., said CD34⁺ cells, express CD3 and CD4 at a lower level than the equivalent number of cells from umbilical cord blood.

In certain embodiments, said placental perfusate cells, e.g., said CD34⁺ cells, comprise fewer CD3⁺ cells than the equivalent number of cells from umbilical cord blood. In certain embodiments, said placental perfusate cells, e.g., said CD34⁺ cells, comprise fewer CD3⁺CD8⁺ cells than the equivalent number of cells from umbilical cord blood. In certain embodiments, said placental perfusate cells, e.g., said CD34⁺ cells, comprise fewer CD3⁺CD4⁺ cells than the equivalent number of cells from umbilical cord blood. In certain embodiments, said placental perfusate or said placental perfusate cells have been depleted of CD3⁺ cells. In certain embodiments, said placental perfusate or said placental perfusate cells have been depleted of CD3⁺CD8⁺ cells. In certain embodiments, said placental perfusate or said placental perfusate cells have been depleted of CD3⁺CD4⁺ cells.

In certain embodiments, the depletion of CD3⁺ cells results in 2-fold fewer CD3⁺ cells than in placental perfusate or placental perfusate cells that have not been depleted. In certain embodiments, the depletion of CD3⁺ cells results in 3-fold fewer CD3⁺ cells than in placental perfusate or placental perfusate cells that have not been depleted. In certain embodiments, the depletion of CD3⁺ cells results in 2, 3, 4, 5, 6, 7, 8, 9, 10, 15, or 20-fold fewer CD3⁺ cells than in placental perfusate or placental perfusate cells that have not been depleted.

In certain embodiments, the depletion of CD3⁺CD8⁺ cells results in 2-fold fewer CD3⁺ CD8⁺ cells than in placental perfusate or placental perfusate cells that have not been depleted. In certain embodiments, the depletion of CD3⁺CD8⁺ cells results in 3-fold fewer CD3⁺CD8⁺ cells than in placental perfusate or placental perfusate cells that have not been depleted. In certain embodiments, the depletion of CD3⁺CD8⁺ cells results in 2, 3, 4, 5, 6, 7, 8, 9, 10, 15, 20, 25, 30, 40 or 50-fold fewer CD3⁺CD8⁺ cells than in placental perfusate or placental perfusate cells that have not been depleted.

In certain embodiments, the depletion of CD3⁺CD4⁺ cells results in 2-fold fewer CD3⁺ CD4⁺ cells than in placental perfusate or placental perfusate cells that have not been depleted. In certain embodiments, the depletion of CD3⁺CD4⁺ cells results in 3-fold fewer CD3⁺CD4⁺ cells than in placental perfusate or placental perfusate cells that have not been depleted. In certain embodiments, the depletion of CD3⁺CD4⁺ cells results in 2, 3, 4, 5, 6, 7, 8, 9, 10, 15, 20, 25, 30, 40 or 50-fold fewer CD3⁺CD4⁺ cells than in placental perfusate or placental perfusate cells that have not been depleted.

In certain embodiments, said placental perfusate cells, e.g., said CD34⁺ cells, comprise fewer CD3⁺CD4⁺CD8⁻CD25^{hi}CD127^{low} cells than the equivalent number of cells from umbilical cord blood. In certain embodiments, said placental perfusate cells, e.g., said CD34⁺ cells, comprise fewer CD3⁺CD4⁺CD8⁻CD25^{hi}CD127^{low} CD45RA⁺ cells than the equivalent number of cells from umbilical cord blood. In certain embodiments, said placental perfusate cells, e.g., said CD34⁺ cells, comprise fewer CD3⁺CD4⁺CD8⁻CD25^{hi}CD127^{low}/CD45RA⁻ cells than the equivalent number of cells from umbilical cord blood. In certain embodiments, said placental perfusate cells, e.g., said CD34⁺ cells, comprise fewer CD3⁺CD4⁺CD8⁻ CD25hiCD127lowCD45RA⁻HLADR⁺ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-} cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-}CD45RA⁺HLADR⁻ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-} CD45RA⁻CCR7⁺ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-}CD45RA⁺CCR7⁻ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-}CD45RA⁺CCR7⁻ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-} CD45RA⁻HLADR⁺ cells than the equivalent number of cells from umbilical cord blood. In certain embodiments, said placental perfusate cells, e.g., said CD34⁺ cells, comprise fewer CD3⁺CD4⁺CD8⁻CD25^{+/-}CD127^{+/-}CD45RA⁻CD69⁺ cells than the equivalent number of cells from umbilical cord blood. In certain embodiments, said placental perfusate cells, e.g., said CD34⁺ cells, comprise fewer CD3⁺CD4⁻CD8⁺ cells than the equivalent number of cells from umbilical cord blood. In certain embodiments, said placental perfusate cells, e.g., said CD34⁺ cells, comprise fewer CD3⁺CD4⁻CD8⁺CD45RA⁺HLADR⁻CCR7⁺ cells than the equivalent number of cells from umbilical cord blood. In certain embodiments, said placental perfusate cells, e.g., said CD34⁺ cells, comprise fewer CD3⁺CD4⁻CD8⁺CD45RA⁻CCR7⁺ cells than the equivalent number of cells from umbilical cord blood. In certain embodiments, said placental perfusate cells, e.g., said CD34⁺ cells, comprise fewer CD3⁺CD4⁻CD8⁺CD45RA⁺CCR7⁻ cells than the equivalent number of cells from umbilical cord blood. In certain embodiments, said placental perfusate cells, e.g., said CD34⁺ cells, comprise fewer CD3⁺CD4⁻CD8⁺CD45RA⁻CCR7⁻ cells than the equivalent number of cells from umbilical cord blood. In certain embodiments, said placental perfusate cells, e.g., said CD34⁺ cells, comprise fewer CD3⁺CD4⁻CD8⁺CD45RA⁻HLADR⁺ cells than the equivalent number of cells from umbilical cord blood. In certain embodiments, said placental perfusate cells, e.g., said CD34⁺ cells, comprise fewer CD3⁺CD4⁺CD8⁺ cells than the equivalent number of cells from umbilical cord blood. In certain embodiments, said placental perfusate cells, e.g., said CD34⁺ cells, comprise fewer CD3⁺CD4-CD8⁻ cells than the equivalent number of cells from umbilical cord blood. In certain embodiments, said placental perfusate cells, e.g., said CD34⁺ cells, comprise fewer CD3⁺CD4-CD8⁻CD69⁺ cells than the equivalent number of cells from umbilical cord blood.

In certain embodiments, any of the CD34⁺ cells described herein, or populations of CD34⁺ cells, are expanded. In certain embodiments, any of the CD34⁺ cells described herein, or populations of CD34⁺ cells, are enriched. In certain embodiments, any of the CD3⁺ cells described herein, e.g. CD34⁺CD3⁺ cells, are depleted.

In certain embodiments, said placental perfusate or said placental perfusate cells have been treated to suppress proliferation of CD3⁺ cells. In certain embodiments, said placental perfusate or said placental perfusate cells have been treated to suppress proliferation of CD3⁺CD8⁺ cells. In certain embodiments, said placental perfusate or said placental perfusate cells have been treated to suppress proliferation of CD3⁺CD4⁺ cells. In a specific embodiment, suppression of proliferation of CD3⁺CD4⁺ cells is accomplished by the addition of isolated CD3⁺CD4⁺CD8⁻CD25^{hi}CD127^{low} cells.

Placental perfusate, placental perfusate cells, and any populations and subpopulations thereof, may be combined. In one embodiment, one or more populations or subpopulations of said placental perfusate cells are combined with total nucleated cells from placental perfusate. In another embodiment, one or more populations or subpopulations of said placental perfusate cells are combined with each other. In a specific embodiment, said one or more populations or subpopulations have been enriched for one or more particular phenotypes of cells. In another specific embodiment, said one or more populations or subpopulations have been isolated from placental perfusate or placental perfusate cells. In another specific embodiment, said one or more populations or subpopulations have been obtained through one or more rounds of cell sorting. In another specific embodiment, said one or more populations or subpopulations have been depleted of one or more particular phenotypes of cells.

In yet another embodiment, a population of placental perfusate or perfusate cells is combined with a plurality of CD34⁺ cells. Such CD34⁺ cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like. In a specific embodiment, the hematopoietic stem cells are CD34⁺ placental endothelial progenitor cells.

In one aspect, provided herein is a method for treating an individual having a central nervous system injury, disease or disorder, comprising administering to the individual placental perfusate or any of the cell populations or subpopulations presented herein, or any combination thereof, in an amount sufficient to produce a detectable improvement in, or reduction in the worsening of, one or more symptoms of the central nervous system injury, disease or disorder. In a specific embodiment, the central nervous system injury, disease, or disorder is ischemic encephalopathy (e.g., hypoxic ischemic encephalopathy). In another specific embodiment, said placental perfusate cells are total nucleated cells from placental perfusate. In another embodiment, said placental perfusate cells are any population, subpopulation, or combination comprising placental perfusate cells described herein. In another specific embodiment, said population of placental perfusate cells comprises placental perfusate cells isolated from perfusion of a single placenta. In another specific embodiment, said population of placental perfusate cells comprises isolated CD34⁺ cells not isolated from said perfusate. In a more specific embodiment, said CD34⁺ cells are isolated from placenta. In another more specific embodiment, said CD34⁺ cells are isolated from umbilical cord blood, placental blood, peripheral blood, or bone marrow. In another more specific embodiment, said CD34⁺ cells express a higher level of Nestin than an equivalent number of CD34⁺ cells from umbilical cord blood.

In another aspect, provided herein are methods of administering HPCs, e.g. human placental perfusate, to a subject, e.g. a human subject, to reduce the severity of graft versus host disease and/or to treat or ameliorate symptoms of metabolic and hematologic disorders, such as hematologic malignancies.

In another aspect, provided herein are methods of administering HPCs, e.g. human placental perfusate, to a subject, e.g. a human subject, to treat or ameliorate symptoms of sarcopenia. Further embodiments of such methods are described in detail in the following sections.

5.2 Isolation, Sorting, and Characterization of Placental Perfusate Cells

Provided herein are methods of obtaining placental perfusate and placental perfusate cells from a mammalian placenta. In all of the embodiments described herein, the preferred perfusate is human placental perfusate, and the preferred perfusate cells are human placental perfusate cells. Also described herein are methods for isolating cell populations and subpopulations, and for characterizing cell populations and subpopulations and combinations thereof.

Mononuclear cells from human placental perfusate (HPCs), e.g., human placental perfusate, for use in accordance with the present disclosure may be collected in any medically or pharmaceutically-acceptable manner and may be present in a composition, e.g., a pharmaceutical composition. In certain embodiments, a composition (e.g., a pharmaceutical composition, i.e., a pharmaceutical grade solution suitable for administration to a human) provided herein comprises human placental perfusate. In certain embodiments, the composition comprises human placental perfusate obtained from partially exsanguinated placenta. In certain embodiments, the composition comprises human placental perfusate obtained from exsanguinated placenta. In certain embodiments, the composition comprises cells, such as stem cells, isolated from human placental perfusate. In certain embodiments, the composition comprises nucleated cells isolated from human placental perfusate, e.g., mononuclear cells or total nucleated cells.

In one embodiment, the HPCs, e.g., human placental perfusate, are sterile.

In a specific embodiment, HPCs or human placental perfusate are processed by removal of red blood cells and/or granulocytes according to standard methods to produce a population of nucleated cells. Such enriched populations of cells may be used unfrozen, or may be frozen for later use. If the population of cells is to be frozen, a standard cryopreservative (e.g., DMSO, glycerol, Epilife™ Cell Freezing Medium (Cascade Biologics) can be added to the enriched population of cells before it is frozen.

In certain embodiments, cells obtained from placental perfusate comprise mononuclear cells from placental perfusate. In certain embodiments, cells obtained from placental perfusate comprise total nucleated cells from placental perfusate. In particular embodiments, perfusate can be processed to remove or substantially remove erythrocytes by addition of hetastarch (hydroxyethyl starch) to the perfusate followed by settling out by gravity.

In certain embodiments, the cells obtained from placental perfusate are obtained from a single placenta. In certain embodiments, the cells obtained from placental perfusate are obtained from more than one placenta. In certain embodiments, the cells obtained from placental perfusate are obtained from two placentas. In embodiments wherein the cells are obtained from greater than one placenta, the cells from the different placentas need not be related or matched to each other.

As described herein, placental perfusate may be obtained from a placenta that has been drained of cord blood and perfused to remove residual blood, prior to perfusion to obtain placental cells. Placental perfusate may be obtained from a placenta that has been drained of cord blood but not perfused to remove residual blood. Placental perfusate may be obtained from a placenta that has been separated from all but 0.5-6.0 inches, e.g., 0.5-1.0, 1.0-1.5, 1.5-2.0, 2.0-2.5, 2.5-3.0, 3.0-3.5, 3.5-4.0, or 4.0-6.0 inches, of the umbilical cord, wherein the umbilical cord may contain residual cord blood, a portion of which may enter the placental perfusate during perfusion and thus is comprised in the placental perfusate. Placental perfusate may be obtained from a placenta that has neither been drained of cord blood nor perfused to remove residual blood. In the latter two embodiments, the placental cells, e.g., nucleated cells from placental perfusate, for example, HPCs, comprise nucleated cells from placental blood and/or cord blood. In a specific embodiment, placental perfusate used in accordance with the present disclosure is free of umbilical cord blood. In another specific embodiment, placental perfusate used in accordance with the present disclosure is substantially free of umbilical cord blood, e.g., said placental perfusate comprises less than 10%, less than 5%, less than 1%, less than 0.5%, or less than 0.1% cord blood. Generally, where cells from perfusate comprise cord blood cells, such cells are considered part of the HPC population, not part of the HT cells, for example, UCB cells, for purposes of the methods provided herein.

Placental perfusate may be collected from a single individual (i.e., as a single unit) for administration, or may be pooled with other units, e.g., from the same individual or from one or more other individuals. In certain embodiments, the placental perfusate or cells obtained therefrom is stored prior to administration. In certain embodiments, a unit of placental perfusate contains a sufficient number of cells such that at least about 1.0×10⁵, 0.5×10⁶, 1.0×10⁶, 1.5×10⁶, 2.0×10⁶, 2.5×10⁶, 3.0×10⁶, 4.0×10⁶, 5.0×10⁶, or 1.0×10⁷ cells obtained from placental perfusate, e.g., total nucleated cells, per kilogram body weight of a subject are administered. In certain embodiments, one unit of placental perfusate or cells obtained therefrom is administered. In certain embodiments, less than one unit is administered. In certain embodiments, more than one unit is administered.

Placentas for obtaining placental perfusate can be recovered following successful birth and placental expulsion. In certain embodiments, the placenta is from a full-term birth. In certain embodiments, the placenta is from a premature birth. In some embodiments, the placenta is the placenta of an infant born at about 23 to about 25 weeks of gestation. In some embodiments, the placenta is the placenta of an infant born at about 26 to about 29 weeks of gestation. In some embodiments, the placenta is the placenta of an infant born at about 30 to about 33 weeks of gestation. In some embodiments, the placenta is the placenta of an infant born at about 34 to about 37 weeks of gestation. In some embodiments, the placenta is the placenta of an infant born at about 37 to about 42 weeks of gestation.

In particular embodiments, the placenta may be stored for a period of about 1 hour to about 72 hours or about 4 to about 24 hours, prior to perfusing the placenta to remove any residual cord blood, or prior to perfusing the placenta without removal of residual cord blood. The placenta can be stored in an anticoagulant solution at a temperature of about 5° C. to about 25° C., e.g., at about room temperature. Suitable anticoagulant solutions are well known in the art. For example, a solution of heparin or warfarin sodium can be used. In one embodiment, the anticoagulant solution comprises a solution of heparin (1% w/w in 1:1000 solution). In certain embodiments, the placenta is stored for no more than 36 hours before HPCs, e.g., human placental perfusate, are collected.

Human placental perfusate or cells obtained therefrom for use in accordance with the present disclosure are generally unrelated to the subject recipient of the cells. Human placental perfusate or cells obtained therefrom for use in accordance with the present disclosure are generally unmatched or partially unmatched to the subject recipient of the cells.

Human placental perfusate or cells obtained therefrom for use in accordance with the present disclosure can be obtained by any method. Placental perfusate can be obtained, e.g., as disclosed in U.S. Pat. Nos. 7,045,148, 7,255,879, and/or 8,057,788, the contents of each of which are incorporated herein by reference in their entirety. Such perfusion can, e.g., be perfusion by the pan method, wherein perfusion liquid is forced through the placental vasculature and perfusion fluid that exudes from the placenta, typically the maternal side, is collected in a pan containing the placenta. Perfusion can also, e.g., be a closed-circuit perfusion, wherein perfusion fluid is passed through, and collected from, only the fetal vasculature of the placenta. See, e.g., U.S. Pat. No. 8,057,788, the contents of which are incorporated herein by reference in their entirety. In a specific embodiment, such perfusion can be continuous, that is, perfusion fluid that has been passed through the placenta is passed through a second time, or a plurality of times, prior to isolation of cells obtained from placental perfusate (e.g., HPCs or total nucleated cells from placental perfusate).

In certain embodiments, about 0.5-2 liters of perfusion fluid, for example, about 0.5-1 liters, or about 750 mL, is used to perfuse a placenta. In specific embodiments, perfusion of the placenta is completed within about 15 minutes to 2 hours, for example, about 30 minutes to 1.5 hours, about 30 minutes to 1 hour, or about 30 minutes.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough RNA for a particular marker to be detectable by RT-PCR, or significantly more RNA for a particular marker than an adult cell, the cell is positive for that marker. Antibodies to cell surface markers (e.g., CD markers such as CD34) and the sequence of specific genes are well-known in the art.

In another embodiment, placental cells, e.g., placental perfusate or perfusate cells can be identified and characterized by a colony forming unit assay. Colony forming unit assays are commonly known in the art.

Placental perfusate or perfusate cells can additionally be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Cells may, for example, be sorted, e.g., sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In another embodiment, magnetic beads can be used to separate or sort cells, and/or to deplete a population of cells. The cells may, for example, be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Placental perfusate cells can be separated using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

5.3 Method of Using Hematopoietic Cells, e.g., Umbilical Cord Blood Cells, and Cells from Human Placental Perfusate

In one aspect, provided herein are methods of transplanting hematopoietic cells to a subject, e.g., a human subject, comprising administering the hematopoietic cells in combination with mononuclear cells from human placental perfusate (HPCs), e.g., human placental perfusate. Said HPCs may be human placental perfusate, total nucleated cells from placental perfusate, or any population, subpopulation, or combination of mononuclear cells from human placental perfusate described herein, including those enriched for or depleted of a particular population or subpopulation. Sources of hematopoietic cells that can be used in the methods of transplanting hematopoietic cells described herein include, for example, bone marrow or cells therefrom, peripheral blood or cells therefrom, and umbilical cord blood or cells therefrom. As used herein, these sources of hematopoietic cells are collectively referred to as “HT cells.”

In one embodiment, provided herein is a method of transplanting HT cells, for example, human umbilical cord blood cells (UCB) cells, e.g., human umbilical cord blood, to a subject, e.g., a human subject, comprising administering the HT cells, for example, human umbilical cord blood cells (UCB) cells, e.g., human umbilical cord blood, in combination with mononuclear cells from human placental perfusate (HPCs), e.g., human placental perfusate. In one embodiment, the HT cells, for example, human UCB cells, e.g., human UCB, are not related to the subject. In a particular embodiment, the HT cells, for example, UCB cells, e.g., human UCB, are partially unmatched to the subject. In another embodiment, the HPCs, e.g., human placental perfusate, are not related to the subject. In a particular embodiment, the HPCs, e.g., human placental perfusate, are partially unmatched to the subject. In another particular embodiment, the HPCs, e.g., human placental perfusate, are not matched to the subject. In yet another embodiment, the HT cells, for example, human UCB cells, e.g., human UCB, are unrelated to the subject and the HPCs, e.g., human placental perfusate, are unrelated to the subject. In still another embodiment, the HT cells, for example, human UCB cells, e.g., human UCB, are unrelated and partially unmatched to the subject and the HPCs, e.g., human placental perfusate, are unrelated and partially unmatched or unmatched to the subject. In one embodiment HPCs, e.g., human placental perfusate, are unrelated and unmatched to the HT cells, for example, human UCB cells, e.g., UCB. In one embodiment HPCs, e.g. human placental perfusate, are unrelated and unmatched to the HT cells, for example, human UCB cells, e.g., UCB, and the recipient.

Unless otherwise noted, “related,” as used herein in the context of UCB or HPCs, refers to self, or to a first or second degree blood relative. For example, UCB that is related to the subject refers to UCB from the subject itself, or from a first or second degree blood relative of the subject. In another example, UCB that is related to HPC refers to UCB and HPC that are from the same donor, or donors that are first or second degree blood relatives. Likewise, unless otherwise noted, “unrelated,” in these contexts, refers to relationships that are more distant than that of a second degree blood relative.

Unless otherwise noted, “matched,” as used herein in the context of UCB or cells from human placental perfusate (e.g., HPCs), refers to HLA matched. In addition, as used herein, “partially unmatched,” as used herein in the context of UCB or cells from human placental perfusate (e.g., HPCs), refers to situations where there is matching at 3/6, 4/6, or 5/6 HLA loci. Also, unless otherwise noted, “unmatched,” as used herein in the context of UCB or cells from human placental perfusate (e.g., HPCs), refers to matching at 0/6, 1/6, or 2/6 HLA loci. “Matched,” “partially unmatched,” and “unmatched” can, for example, refer to the relationship between the HT cells, for example, UCB cells, and HPCs, between units of HT cells, for example, UCB cells, and/or between the HT cells, for example, UCB cells, and/or HPCs and the subject that is the recipient of the cells.

In certain embodiments, such methods comprise administering one unit of UCB, or cells therefrom. In another embodiment, the methods presented herein comprise administering multiple units of UCB, or cells therefrom. For example, the methods presented herein can comprise administering two, three, or four units of UCB, or cells therefrom. In instances wherein greater than one unit of HT cells, for example, UCB cells, is used, in certain embodiments, at least a portion of the HT cells, for example, UCB cells, can be unrelated to the subject, to the HPCs, and/or to other portions of the HT cells, for example, UCB cells (e.g., other UCB cell units). In instances wherein greater than one unit of HT cells, for example, UCB cells, is used, in certain embodiments, at least a portion of the HT cells, for example, UCB cells, can be unmatched or partially unmatched to the subject, to the HPCs, and/or to other portions of the HT cells, for example, UCB cells (e.g., other UCB cell units). In another embodiment, the methods presented herein can comprise administering less than one unit of HT cells or UCB, or cells therefrom. For example, the methods presented herein can comprise administering 0.2, 0.3, 0.4, 0.5, 0.6, 0.7, 0.8, or 0.9 units of HT cells or UCB, or cells therefrom. In particular embodiments, the methods presented herein can comprise administering a particular number of units (less than one, one, or more than one) over multiple administrations.

In another aspect, provided herein are methods for inducing chimerism in a subject, comprising administering to the subject a combination of HT cells, for example, UCB cells, e.g., UCB, and HPCs, e.g., human placental perfusate, wherein at least a portion of the HT cells, for example, UCB cells, are partially unmatched to the subject, and/or the HPCs are unmatched or partially unmatched to the subject, such that chimerism in the subject occurs. “Chimerism,” unless noted otherwise, as used herein, refers to the presence in a subject of non-self DNA, e.g., the presence of DNA from cells that are unmatched or partially unmatched relative to the recipient subject.

In one embodiment of such methods, greater than one unit of HT cells, for example, UCB cells, is administered to the subject, e.g., 2, 3, or 4 units of HT cells, for example, UCB cells, are administered to the subject. In particular embodiments wherein greater than one unit of HT cells, for example, UCB cells, is administered to the subject the method of inducing chimerism can result in multiple chimerism, that is, chimerism involving greater than one, and up to all, of the administered HT cell, e.g., UCB cell, units, or progeny thereof, can result.

In another embodiment of such methods, chimerism involving the HPCs or progeny thereof can result. In yet another embodiment, chimerism involving the HT cells, for example, UCB cells (including multiple chimerism in instances wherein greater than one unit of HT cells, for example, UCB cells, is administered), or progeny thereof, and the HPCs, or progeny thereof, can result.

In still yet another embodiment of such methods, the HT cells, for example, UCB cells, are unrelated to the subject. In instances in which greater than one unit of HT cells, e.g., UCB, is administered, one or more of the HT cell, e.g., UCB cell, units can be unrelated to the subject. In a particular embodiment of such methods, the HPCs are unrelated to the subject and can, additionally, be unrelated to the HT cells, for example, UCB cells. In still another embodiment of such methods, both the HT cells, for example, UCB cells, and the HPCs are unrelated to the subject.

In certain embodiments of such methods, chimerism (comprising either or both HT cells, for example, UCB cells, or progeny thereof, or HPCs, or progeny thereof) is first detected in the subject within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 days, or more of administration of the HT cells, for example, UCB cells, in combination with the HPCs to the subject.

Chimerism can be detected using methods known in the art. For example, chimerism can be detected using blood samples. In one embodiment, chimerism is detected using a polymerase chain reaction (PCR)-based method, e.g., by short tandem repeat assays. In one embodiment, a test for chimerism after a hematopoietic stem cell transplant involves identifying the genetic profiles of the recipient and of the donor and then evaluating the extent of mixture in the recipient's blood, bone marrow, or other tissue. Chimerism testing (engraftment analysis) by DNA employs methodology commonly used in human identity testing and is accomplished by the analysis of genomic polymorphisms called short tandem repeat (STR) loci. In one embodiment, quantitation (e.g., using short tandem repeat assays) of peripheral blood donor chimerism (UCB/s and perfusate cells)(whole blood, NK and T Cell) is assessed on Days 7, 14, 30, 60, 100 and 180 (+/−10 days), with quantitation (e.g., using short tandem repeat assays) of peripheral blood recipient chimerism assessed at baseline along with chimerism of the donor cells (UCB and perfusate cells) at baseline.

In still another aspect, provided herein are methods for cell engraftment in a subject, comprising administering to the subject a combination of HT cells, for example, human UCB cells, e.g., UCB, and HPCs, e.g., human placental perfusate, wherein at least a portion of the HT cells, for example, UCB cells, are partially matched to the subject, and/or the HPCs are unmatched or partially unmatched to the subject, such that cell engraftment in the subject occurs. In certain embodiments, the cell engraftment comprises engraftment of HT cells, for example, UCB cells, or progeny thereof. In certain other embodiments, the cell engraftment comprises engraftment of HPCs, or progeny thereof. In still other embodiments, the engraftment comprises engraftment of HT cells, for example, UCB cells, or progeny thereof, and HPCs, or progeny thereof.

In one embodiment of such methods, the HT cells, for example, UCB cells, are unrelated to the subject. In a particular embodiment, the HT cells, for example, UCB cells, are partially unmatched to the subject. In another particular embodiment, the HPCs are unrelated to the subject and can, additionally, be unrelated to the HT cells, for example, UCB cells. In a particular embodiment, the HPCs are partially unmatched to the subject. In another particular embodiment, the HPCs are not matched to the subject. In yet another embodiment, the UCB cells are unrelated to the subject and the HPCs are unrelated to the subject. In still another embodiment, the HT cells, for example, UCB cells, are unrelated and partially unmatched to the subject and the HPCs are unrelated and partially unmatched or unmatched to the subject. In certain embodiments, the methods presented herein exhibit an enhanced ability to engraft as compared to administration of HT cells, for example, UCB cells, alone.

Engraftment can be detected using methods known in the art. For example, in one embodiment, a complete blood count with differential may performed every 1-3 days from Day 0 to absolute neutrophil count >500/mm3 for 3 days after nadir is reached and until platelet count reaches ≥20,000/mm³ for 3 consecutive measurements on 3 different days and independence from platelet transfusion for a minimum of 7 days. As used herein, “neutrophil engraftment” refers to the first of three days following the neutrophil nadir with an absolute neutrophil count above 500/mm³. As used herein, “platelet engraftment” refers to the first of three consecutive days demonstrating a platelet count ≥20,000/mm³, after a seven day period of platelets ≥20,000/mm³ without transfusions.

In certain embodiments, cell engraftment in the subject is detected within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 days, or 2 months, 2.5 months, 3 months, or more of administration of the HT cells, for example, UCB cells, in combination with HPCs to the subject.

In certain embodiments, the methods presented herein comprise administering one unit of HT cells, for example, UCB cells, e.g., UCB. In another embodiment, the methods presented herein comprise administering multiple units of HT cells, for example, UCB cells, e.g., UCB. For example, the methods presented herein can comprise administering two, three, or four units of HT cells, for example, UCB cells, e.g., UCB.

In still another aspect, provided herein are methods for reducing the duration or severity of GVHD in a subject, comprising administering to the subject a combination of HT cells, for example, human UCB cells, e.g., UCB, and HPCs, e.g., human placental perfusate, wherein at least a portion of the HT cells, e.g., UCB cells, are partially matched to the subject, and/or the HPCs are unmatched or partially unmatched to the subject, such that a reduction in the duration or severity of GVHD in the subject occurs.

In one embodiment of such methods, the HT cells, for example, UCB cells, are unrelated to the subject. In a particular embodiment, the HT cells, for example, UCB cells, are partially unmatched to the subject. In another particular embodiment, the HPCs are unrelated to the subject and can, additionally, be unrelated to the HT cells, for example, UCB cells. In a particular embodiment, the HPCs are partially unmatched to the subject. In another particular embodiment, the HPCs are not matched to the subject. In yet another embodiment, the UCB cells are unrelated to the subject and the HPCs are unrelated to the subject. In still another embodiment, the HT cells, for example, UCB cells, are unrelated and partially unmatched to the subject and the HPCs are unrelated and partially unmatched or unmatched to the subject. In certain embodiments, the methods presented herein exhibit reduced severity or duration of GVHD as compared to administration of HT cells, for example, UCB cells, alone.

In certain embodiments, the methods presented herein comprise administering one unit of HT cells, for example, UCB cells, e.g., UCB. In another embodiment, the methods presented herein comprise administering multiple units of HT cells, for example, UCB cells, e.g., UCB. For example, the methods presented herein can comprise administering two, three, or four units of HT cells, for example, UCB cells, e.g., UCB.

In another aspect, provided herein are methods for treating an individual having sarcopenia, comprising administering to the individual placental perfusate or any of the cell populations or subpopulations presented herein, or any combination thereof, in an amount sufficient to produce a detectable improvement in, or reduction in the worsening of, one or more symptoms of sarcopenia, comprising administering to the subject a combination of HT cells, for example, human UCB cells, e.g., UCB, and HPCs, e.g., human placental perfusate.

In still another aspect, provided herein are methods for treating an individual having a central nervous system injury, disease or disorder, comprising administering to the individual placental perfusate or any of the cell populations or subpopulations presented herein, or any combination thereof, in an amount sufficient to produce a detectable improvement in, or reduction in the worsening of, one or more symptoms of the central nervous system injury, disease or disorder, comprising administering to the subject a combination of HT cells, for example, human UCB cells, e.g., UCB, and HPCs, e.g., human placental perfusate. In a specific embodiment, the central nervous system injury, disease, or disorder is ischemic encephalopathy (e.g., hypoxic ischemic encephalopathy).

In certain embodiments, the methods presented herein comprise administering HT cells, for example, UCB cells, e.g., UCB, concurrently with the HPCs, e.g., human placental perfusate. In a particular embodiment, the cells are administered to a subject simultaneously. In another embodiment, the HT cells, for example, UCB cells, and HPCs are administered to the subject within 0.5, 1, 1.5, 2, 3, 4, 6, 8, 10, 12, 16, 18, or 24 hours or more, or within 1, 2, 3, 4, 5, 6, or 7 days or more of each other. In a specific embodiment, the HT cells, for example, UCB cells, e.g., UCB, is administered to the subject, then the HPC, e.g., human placental perfusate, is administered, e.g., is administered within 1 hour of administration of UCB, or within the minimum period necessary to verify that the subject is not exhibiting an adverse reaction to the UCB administration.

The methods provided herein can exhibit advantages that can include, for example, a reduction in the length of time to cell engraftment, limiting the time the subject is neutropenic, limiting the time the subject is thrombocytopenic, establishment of chimerism, and reducing the severity or duration of, or preventing, GVHD, relative to administration of HT cells, for example, UCB cells, e.g., UCB, alone.

The ratio of HT cells, for example, UCB cells, and HPCs administered can vary. The ratio of HT cells, for example, UCB cells, and HPCs can be determined according to the judgment of those of skill in the art. In certain embodiments, the ratio of HT cells, for example, UCB cells, to HPCs is about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000. In certain embodiments, the ratio of HT cells, for example, UCB cells, to HPCs is between about 20:1 and about 1:20, or is about 1:10, about 1:5, about 1:1, about 5:1 or about 10:1.

Administration of HT cells, for example, UCB cells, and HPCs can be performed using any technique for cell administration known in the art. In one embodiment, administration is venous, for example, intravenous, e.g., through an IV, PICC line, central line, etc. For example, HT cells, for example, UCB cells, and HPCs may be administered, in separate compositions or in a single composition, to a subject in any pharmaceutically or medically acceptable manner, including by injection or transfusion. In certain embodiments, the composition(s) may be formulated as an injectable composition (e.g., WO 96/39101, incorporated herein by reference in its entirety).

In certain embodiments, HT cells, for example, UCB cells, or HPCs are administered to a subject parenterally. The term “parenteral” as used herein includes subcutaneous injections, intravenous, intramuscular, intra-arterial injection, or infusion techniques. In certain embodiments, HT cells, for example, UCB cells, or HPCs are administered to a subject intravenously. In certain other embodiments HT cells, for example, UCB cells, or HPCs are administered to a subject intraventricularly.

HT cells, for example, UCB cells, and HPCs may be contained, separately or together, in any pharmaceutically-acceptable carrier. The HT cells, for example, UCB cells, or HPCs may be carried, stored, or transported in any pharmaceutically or medically acceptable container, for example, a blood bag, transfer bag, plastic tube, syringe, vial, or the like.

Administration of HT cells, for example, UCB cells, and/or HPCs to a subject can be performed once or a plurality of times. In certain embodiments, administration is performed once. In certain embodiments, administration is performed a plurality of times, e.g., two, three, four, or more times. In certain embodiments, HT cells, for example, UCB cells, are administered a plurality of times. In certain embodiments, HPCs are administered a plurality of times.

In certain embodiments, the amount of cord blood or cells obtained therefrom (e.g., total nucleated cells from umbilical cord blood) administered to a subject in accordance with the methods described herein can be determined based on the number of cells present in the cord blood. The amount or number of UCB or cells obtained therefrom (e.g., total nucleated cells from umbilical cord blood) and/or human placental perfusate or HPCs or total nucleated cells obtained therefrom administered to the subject depends on the source of umbilical cord blood or cells obtained therefrom (e.g., total nucleated cells from umbilical cord blood) and/or human placental perfusate or HPCs or total nucleated cells obtained therefrom, the severity or nature of disorders or conditions to be treated, as well as age, body weight and physical condition of the subject, etc. In certain embodiments, about 0.01 to about 0.1, about 0.1 to about 1, about 1 to about 10, about 10 to about 10², about 10² to about 10³, about 10³ to about 10⁴, about 10⁴ to about 10⁵, about 10⁵ to about 10⁶, about 10⁶ to about 10⁷, about 10⁷ to about 10⁸, or about 10⁸ to about 10⁹ umbilical cord blood cells (e.g., total nucleated cells from umbilical cord blood), human placental perfusate or cells obtained therefrom (e.g., HPCs or total nucleated cells from placental perfusate), or total umbilical cord blood cells and cells obtained from placental perfusate (e.g., HPCs or total nucleated cells) per kilogram body weight of a subject are administered. In various embodiments, at least about 0.1, 1, 10, 10², 10³, 10⁴, 10⁵, 10⁶, 10⁷, 10⁸, or 10⁹ umbilical cord blood cells (e.g., total nucleated cells from umbilical cord blood), cells obtained from placental perfusate (e.g., HPCs or total nucleated cells from placental perfusate), or umbilical cord blood cells and cells obtained from placental perfusate per kilogram body weight of a subject are administered.

In specific embodiments, at least about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood), cells obtained from placental perfusate (e.g., HPCs or total nucleated cells from placental perfusate), or umbilical cord blood cells and cells obtained from placental perfusate (e.g., HPCs or total nucleated cells from placental perfusate) per kilogram body weight of a subject are administered. In a more specific embodiment, at least about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, or 5.0×10⁶ cells obtained from placental perfusate (e.g., HPCs or total nucleated cells from placental perfusate) per kilogram body weight of a subject are administered. In a more specific embodiment, at least about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood) per kilogram body weight of a subject are administered. In various embodiments, at most about 10⁴, 10⁵, 10⁶, 10⁷, 10⁸, or 10⁹ umbilical cord blood cells, cells obtained from placental perfusate (e.g., HPCs or total nucleated cells from placental perfusate), or umbilical cord blood cells and cells obtained from placental perfusate (e.g., HPCs or total nucleated cells from placental perfusate) per kilogram body weight of a subject are administered. In specific embodiments, at most about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood), cells obtained from placental perfusate (e.g., HPCs or total nucleated cells from placental perfusate), or umbilical cord blood cells and cells obtained from placental perfusate (e.g., HPCs or total nucleated cells from placental perfusate) per kilogram body weight of a subject are administered. In a more specific embodiment, at most about 0.5×10⁶, 1.0×10⁶, 1.5×10⁶, 2.0×10⁶, 2.5×10⁶, 3.0×10⁶, 3.5×10⁶, 4.0×10⁶, 4.5×10⁶, or 5.0×10⁶ cells obtained from placental perfusate (e.g., HPCs or total nucleated cells from placental perfusate) per kilogram body weight of a subject are administered. In a more specific embodiment, at most about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood) per kilogram body weight of a subject are administered.

In specific embodiments of the above embodiments, the cord blood cells (e.g., total nucleated cells from umbilical cord blood) or cells obtained from placental perfusate (e.g., total HPCs or nucleated cells from placental perfusate) are CD34+ cells. In certain embodiments, at least about 10⁴ to about 10⁷ CD34+ cells per kilogram body weight are administered. Such CD34+ cells can be from cord blood alone, or can be from cord blood and placental perfusate.

The HT cells, for example, UCB cells, e.g., UCB, and HPCs, e.g., placental perfusate, can be delivered in a volume appropriate for the size of the subject. Typical blood volume of a human adult is about 85-100 mL/kg body weight. Thus, the blood volume for human adults ranges from approximately 40 mL to approximately 300 mL. In various embodiments, therefore, HT cells, for example, UCB cells, e.g., UCB, and HPCs, e.g., placental perfusate is administered in a total volume of about 0.5 mL, 1.0 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, or about 30 mL, or more. The administration of such volumes can be a single administration or in multiple administrations. The time over which such volumes of cord blood or number of cord blood cells, or human placental perfusate or cells obtained therefrom (e.g., HPCs or total nucleated cells from placental perfusate) can be administered can vary from, e.g., 0.5 hours, 1 hour, 1.5 hours, 2 hours, 2.5 hours, 3 hours, 3.5 hours, 4 hours, or more.

In certain embodiments, small transfusions under 20 mL are performed using a syringe. Larger-volume transfusions can administered by an infusion device, e.g., within a period of one to four hours.

The methods of provided herein can be performed on any subject in need thereof. In one aspect, the subject is in need of hematopoietic reconstitution, partial reconstitution, or augmentation. In certain embodiments, the subject is a human subject. In certain embodiments, the subject is an adult human subject. In certain embodiments, the subject is 25 years or younger. In certain embodiments, the subject is an infant.

In certain embodiments, prior to the methods presented herein, e.g., methods of transplanting, inducing chimerism and/or methods of engraftment, the subject has been administered myeloablative conditioning, using, e.g., TBI, Clofarabine, and/or Ara-Cl; reduced toxicity conditioning using, e.g., Busulfan, Fludarabine, and/or Alemtuzumab; or radiation therapy or other therapy such as immunosuppressive therapy or a therapy that reduces blood cell count.

In a particular aspect, the methods provided herein can be used as methods for the treatment of a metabolic disorder such as an inborn error of metabolism, adrenoleukodystrophy, mucopolysaccharidosis, Niemann-Pick disease, metachromatic leukodystrophy, Wolman disease, Krabbe's disease, Gaucher's disease, fucosidosis, or Batten disease in a subject in need thereof.

In another particular aspect, the methods provided herein can be used as methods for the treatment of a hematologic disorder or malignancy, e.g., a lymphohematopoietic malignancy, myelodysplastic syndrome, amegakaryocytic thrombocytopenia, leukemias such as acute lymphoblastic leukemia (ALL) and acute myelogenous leukemia (AML), neutropenia, sickle cell disease such as sickle cell anemia, beta thalassemia (e.g. beta thalassemia major), severe combined immunodeficiency disease, marrow failure, or anemia such as severe aplastic anemia or Diamond-Blackfan anemia in a subject in need thereof.

As used herein, the terms “treat,” “treating,” and “treatment” refer to the reduction or amelioration of the progression, severity, and/or duration, of a disorder or condition, or any parameter or symptom of such a disorder or condition. Treatment may be considered efficacious if the subject survives, or if the disorder or condition to be treated is measurably improved in any way as a result of the treatment. Such improvement may be shown by, e.g., one or more measurable indicators including, for example, detectable changes in a physiological condition or set of physiological conditions associated with a particular disease, disorder or condition. Treatment is also considered effective if one or more indicators appear to respond to such treatment by changing to a value that is within, or closer to, a normal value for, e.g. individuals of similar age, than such indicator(s) would be expected to lie in the absence of the treatment.

In certain embodiments of the methods provided herein, the methods provided herein can be used as a first therapy in combination with one or more second therapies in the treatment of a disorder or condition. Such second therapies include, but are not limited to, surgery, hormone therapy, immunotherapy, phototherapy, or treatment with certain drugs. Exemplary therapies that can be used in combination with the methods provided herein include control of environmental temperature; support with oxygen; a respirator or a ventilator; peripheral blood transfusion; iron supplementation; intravenous feeding; phototherapy; surgery; agents for the treatment of metabolic disorders or hematologic disorders (including hematologic tumors); antibiotics or antiviral drugs; anti-inflammatory agents (e.g., steroidal anti-inflammatory compounds, non-steroidal anti-inflammatory (NSAID) compounds); nitric oxide; antihistamines; immune suppressants; and immunomodulatory compounds (e.g., a TNF-α inhibitor).

5.4 Umbilical Cord Blood Cells

Umbilical cord blood (also referred to herein as UCB or “cord blood”) for use in accordance with the present disclosure may be collected in any medically or pharmaceutically-acceptable manner and may be present in a composition, e.g., a pharmaceutical composition. Various methods for the collection of cord blood have been described. See, e.g., U.S. Pat. Nos. 6,102,871; 6,179,819; and 7,147,626, the contents of each of which are incorporated by reference in its entirety. A conventional technique for the collection of cord blood is based on the use of a needle or cannula, which is used with the aid of gravity. Cord blood may be collected into, for example, blood bags, transfer bags, or sterile plastic tubes.

In some embodiments, umbilical cord blood is obtained from a commercial cord blood bank (e.g., LifeBankUSA, etc.). In another embodiments, umbilical cord blood is collected from a post-partum mammalian umbilical cord and used immediately (e.g., within 1, 2, 3, 4, 5, 6, 7, 8, 9, 10, 11, or 12 hours of collection). In other embodiments, the cord blood used to treat a subject is cord blood that has been cryopreserved. Umbilical cord blood can be collected from a single umbilical cord or from a plurality of umbilical cords.

In certain embodiments, the HT cells, for example, UCB cells, are unrelated to the subject and/or the HPCs. In another embodiment, the HT cells, for example, UCB cells, are partially unmatched to the subject and/or the HPCs. In yet another embodiment, the HT cells, for example, UCB cells, are unmatched to the HPCs. In still another embodiment, the HT cells, for example, UCB cells, are unrelated and unmatched to the HPCs. In particular embodiments the UCB is matched to the subject at 3/6, 4/6, or 5/6 HLA loci. In particular embodiments the HT cells, e.g., from an adult source, are matched to the subject at 6/8, 7/8, or 8/8 HLA loci.

In some embodiments, umbilical cord blood is prepared from preterm umbilical cord. In other embodiments, umbilical cord blood is prepared from full-term umbilical cord. In certain embodiments, umbilical cord blood is obtained from a post-partum mammalian umbilical cord of a full-term birth. In other embodiments, umbilical cord blood is obtained from a post-partum mammalian umbilical cord of a premature birth. In some embodiments, the umbilical cord is the umbilical cord of an infant born at about 23 to about 25 weeks of gestation. In some embodiments, the umbilical cord is the umbilical cord of an infant born at about 26 to about 29 weeks of gestation. In some embodiments, the umbilical cord is the umbilical cord of an infant born at about 30 to about 33 weeks of gestation. In some embodiments, the umbilical cord is the umbilical cord of an infant born at about 34 to about 37 weeks of gestation. In some embodiments, the umbilical cord is the umbilical cord of an infant born at about 37 to about 42 weeks of gestation.

Cord blood, or cells obtained therefrom (e.g., total nucleated cells or stem cells derived therefrom), may be collected from a single individual (i.e., as a single unit) for administration, or may be pooled with other units. In certain embodiments, the cord blood, or cells obtained therefrom (e.g., total nucleated cells or stem cells derived therefrom) is stored prior to use. Where umbilical cord blood is pooled from a plurality of umbilical cords, the pooled cord blood can comprise umbilical cord blood from full-term births only, cord blood from a combination of full-term births, or cord blood from premature births only. For example, cord blood from the umbilical cord of a premature infant can be combined with, e.g., cord blood from other premature infants, cord blood from full-term births only, or a combination of cord blood from both premature and full-term placentas. Cord blood, including autologous or allogeneic cord blood, can also be combined with peripheral blood. In certain embodiments, cord blood from premature births is used, as such cord blood comprises relatively high numbers of CD34+ stem cells per unit volume, compared to cord blood from full-term births. In certain embodiments, a unit of cord blood contains a sufficient number of cells such that at least about 1.0×10⁶, 1.5×10⁶, 2.0×10⁶, 1.5×10⁶, 2.0×10⁶, 2.5×10⁶, 3.0×10⁶, 3.5×10⁶, 4.0×10⁶, 4.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ cells obtained from said cord blood, e.g., total nucleated cells from cord blood, per kilogram body weight of a subject are administered. In certain embodiments, one unit of cord blood or cells obtained therefrom is administered. In certain embodiments, less than one unit is administered. In certain embodiments, more than one unit is administered, e.g., two or more (e.g., 2, 3, 4, 5, 6, or more) units are administered.

### 6. EXAMPLES

### 6.1 Example 1: Human Placental Perfusate Cell Composition

This example illustrates the determination of the composition of human placental perfusate by cell type and associated phenotype.

Human placental perfusate (HPP) was obtained as described in Section 5.2, above. Bags of donor matched HPP and human umbilical cord blood (HUCB) were thawed at 37° C. separately, followed by dilution with an equal volume of thawing medium (IMDM (Cat#30-2005, ATCC)+2% FBS (Cat# SH30070.03, Hyclone)+P/S (Cat#15140-122, Gibco)). The diluted cell mixtures were spun at 400 g for 8 min if 15 ml conical tubes were used, 10 min for 50 ml conical tubes. The cell pellet was resuspended at 1×10⁷/ml with FACS buffer (PBS (Cat#10010-023, Gibco)+2% FBS+P/S). RBC (red blood cells) were lysed by adding Ammonium chloride solution (Cat#07850, StemCell) at the ratio of Ammonium chloride to cells as 9:1 on ice for 10 min. After RBC lysis, the samples were spun at 400 g for 5 min, followed by two washes with FACS buffer. The cell pellets were then resuspended with Cytofix/cytoperm solution (Cat#554722, BD Biosciences) at 1 ml per 1×10⁷ cells for 20 minutes at 4° C. The samples were washed two times with FACS buffer, followed by staining with fluorochrome-conjugated antibodies for 20 min in the dark at RT (room temperature). The phenotype panel is listed in Table 1 and Table 2. The information for the antibodies is listed in Table 3. The stained samples were washed two times with FACS buffer and resuspended at 200 μl FACS buffer for data collection: 9-color panel by FACS Aria (BD Biosciences), 6-color panel by FACS Canto II (BD Biosciences) following the instructions provided by manufacturer. Data analysis was done by FlowJo (Tree Star). A paired student T-test was used for statistical analysis.

TABLE 1
9-color phenotype panel

| | FITC | PE | PerCP | APC | PE-CY7 | APC-Cy7( [symbol] | AF700 | BV421/PB | V500 |
|---|---|---|---|---|---|---|---|---|---|
| 1. Blank | | | | | | | | | |
| 2. T cell | CD45RA | CD25 | CD8 | CD69 | CD127 | CD3 | CCR7 | HLA DR | CD4 |
| 3. B cell | IgD | CD24 | CD20 | CD27 | CD38 | CD45 | — | CD19 | CD3 |
| 4. Monocyte/NK | CD83 | CD142 | CD163 | CD14 | CD56 | CD16 | CD3 + CD19 | HLA DR | CD45 |
| 5. DC cell | CD83 | CD86 | CD14 | CD123 | CD11c | CD16 | CD3 + CD19 | HLA DR | CD45 |

[symbol]

indicates data missing or illegible when filed

TABLE 2
6-color phenotype panel.

| | | FITC | PE | PerCP | APC | PE-Cy7 | APC-Cy7 |
|---|---|---|---|---|---|---|---|
| 1. | Blank | | | | | | |
| 2. | Isotype control | mouse IgG1 | mouse IgG1 | mouse IgG1 | mouse IgG1 | mouse IgG1 | mouse IgG1 |
| 3. | EPC | CD31 | KDR | | CXCR4 | CD34 | CD45 |
| 4. | HPC | CD34 | CD38 | | AC133 | CD117 | CD45 |
| 5. | Progenitor | CD45RA (B220) | CD61 | | CD41 | CD34 | CD45 |
| 6. | NPC | Nestin | CD140a | CD45 | AC133 | CD117 | CD34 |
| 7. | MHC | HLA-DR, DP, DQ | HLA-G | CD56 | HLA-ABC | | CD3 |
| 8. | MHC-2 | HLA-A | HLA-B | | HLA-E | | |
| 9. | MSC | CD105 | CD44 | CD34 | CD200 | CD117 | CD10 |
| 10. | MSC-2 | CD105 | SSEA4 | SSEA3 | CD73 | CD44 | |

TABLE 3
Information regarding antibodies used for phenotype characterization.

| Antibodies | Vendor | Cat# |
|---|---|---|
| FITC Mouse IgG1 | BD | 555748 |
| FITC anti-human CD31 | BD | 555445 |
| FITC anti-human CD34 | BD | 555821 |
| FITC anti-human CD45RA | BD | 347723 |
| FITC anti-human Nestin | BD | IC125F |
| FITC anti-human HLA-DR,DP,DQ | BD | 555558 |
| FITC anti-human HLA-A | LS Bio | LS-C24431 |
| FITC anti-human CD105 | BD | 561443 |
| FITC anti-human CD45RA | BD | 556626 |
| FITC anti-human IgD | BD | 555778 |
| FITC anti-human CD83 | Ebioscience | 11-0839-42 |
| PE Mouse IgG1 | BD | 551436 |
| PE anti-human KDR | R&D | LGE2512101 |
| PE anti-human CD38 | BD | 347687 |
| PE anti-human CD61 | BD | 555754 |
| PE anti-human CD140a | BD | 556002 |
| PE anti-human HLA-G | Ebioscience | 12-9957-42 |
| PE anti-human HLA-B | LS Bio | 36621 |
| PE anti-human CD44 | BD | 550989 |
| PE anti-human SSEA4 | BD | 560128 |
| PE anti-human CD25 | BD | 557138 |
| PE anti-human CD24 | BD | 555428 |
| PE anti-human CD142 | BD | 550312 |
| PE anti-human CD14 | BD | 557154 |
| PerCP Mouse IgG1 | BD | 559425 |
| PerCP anti-human CD45 | BD | 555484 |
| PerCP anti-human CD56 | BD | 555517 |
| PerCP anti-human CD34 | BD | 555823 |
| PerCP anti-human SSEA3 | BD | 561564 |
| PerCP anti-human CD8 | Biolegend | 344708 |
| PerCP anti-human CD20 | BD | 347674 |
| PerCP anti-human CD163 | Biolegend | 333608 |
| PerCP anti-human CD86 | Biolegend | 305420 |
| APC Mouse IgG1 | BD | 550854 |
| APC anti-human CXCR4 | BD | 555976 |
| APC anti-human AC133 | MACS | 130-090-826 |
| APC anti-human CD41 | R&D | FAB7616A |
| APC anti-human HLA-ABC | BD | 555555 |
| APC anti-human HLA-E | Ebioscience | 17-9953-42 |
| APC anti-human CD200 | R&D | FAB27241A |
| APC anti-human CD73 | BD | 560847 |
| APC anti-human CD69 | BD | 555533 |
| APC anti-human CD27 | BD | 558664 |
| APC anti-human CD14 | BD | 555399 |
| APC anti-human CD123 | Ebioscience | 17-1239-42 |
| PE-Cy7 Mouse IgG1 | BD | 557646 |
| PE-Cy7 anti-human CD34 | BD | 348791 |
| PE-Cy7 anti-human CD117 | BD | 339195 |
| PE-Cy7 anti-human CD44 | BD | 560533 |
| PE-Cy7 anti-human CD127 | BD | 560822 |
| PE-Cy7 anti-human CD38 | BD | 335790 |
| PE-Cy7 anti-human CD56 | Biolegend | 304628 |
| PE-Cy7 anti-human CD11c | BD | 561356 |
| APC-Cy7 Mouse IgG1 | BD | 557873 |
| APC-Cy7 anti-human CD45 | BD | 557833 |
| APC-Cy7 anti-human CD34 | Biolegend | 343514 |
| APC-Cy7 anti-human CD3 | BD | 557832 |
| APC-Cy7 anti-human CD10 | Biolegend | 312212 |
| APC-Cy7 anti-human CD3 | BD | 641406 |
| APC-Cy7 anti-human CD45 | BD | 641408 |
| APC-Cy7 anti-human CD16 | BD | 560195 |
| BV421/PB anti-human HLA-DR | Biolegend | 307633 |
| BV421/PB anti-human CD19 | BD | 562440 |
| V500 anti-human CD4 | BD | 561488 |
| V500 anti-human CD3 | BD | 561416 |
| V500 anti-human CD45 | BD | 560777 |
| AF700 anti-human CCR7 | BD | 561143 |
| AF700 anti-human CD3 | BD | 557943 |
| AF700 anti-human CD19 | BD | 557921 |

Mononuclear cells from the HPP were analyzed to determine composition of various mononuclear cell types. Table 4 details the cell types identified:

TABLE 4
Composition of Human Placental Perfusate

| | |
|---|---|
| Cell Type | Associated Phenotype |

LEUKOCYTES ~70 TO 90%

| | |
|---|---|
| T lymphocytes | CD3⁺ CD45⁺ (22.51% ± 14.85%) |
| B lymphocytes | CD3⁻ CD19⁺ (10.12% ± 4.88%) |
| Natural Killer cells | CD3⁻ CD56⁺ (6.45% ± 4.08) |
| Monocytes | CD3⁻ CD14⁺ (26.56% ± 5.22%) |
| Granulocytes | CD3⁻ CD11b⁺ |

PROGENITORS

| | |
|---|---|
| Hematopoietic Stem Cells | CD34⁺ (3.65% ± 2.50%), |
| | CD34⁺ CD45⁻ (1.91% ± 1.13%) |
| Endothelial progenitors | CD34⁺ CD31⁺ (2.93% ± 1.87%), |
| | CD34⁺ KDR⁺ (1.63% ± 1.14%), |
| | CD34⁺ CXCR4⁺ (3.28% ± 2.27%) |
| MSC⁻like cells | CD117⁻ CD34⁻ CD105⁺ CD44⁺ |
| | (1.91% ± 1.08%), |
| | CD34⁻ CD10⁺ CD200⁺ CD105⁺ |
| | (0.56% ± 0.71%), |
| | CD105⁺ CD44⁺ CD73⁺ |
| | (2.32% ± 1.45%) |
| Neural progenitors | CD34⁺ Nestin⁺ (2.23% ± 1.75%) |

### 6.2 Example 2: Total Nucleated Cell Count in Human Placental Perfusate

This example illustrates the determination of the total nucleated cell count of human placental perfusate and umbilical cord blood units.

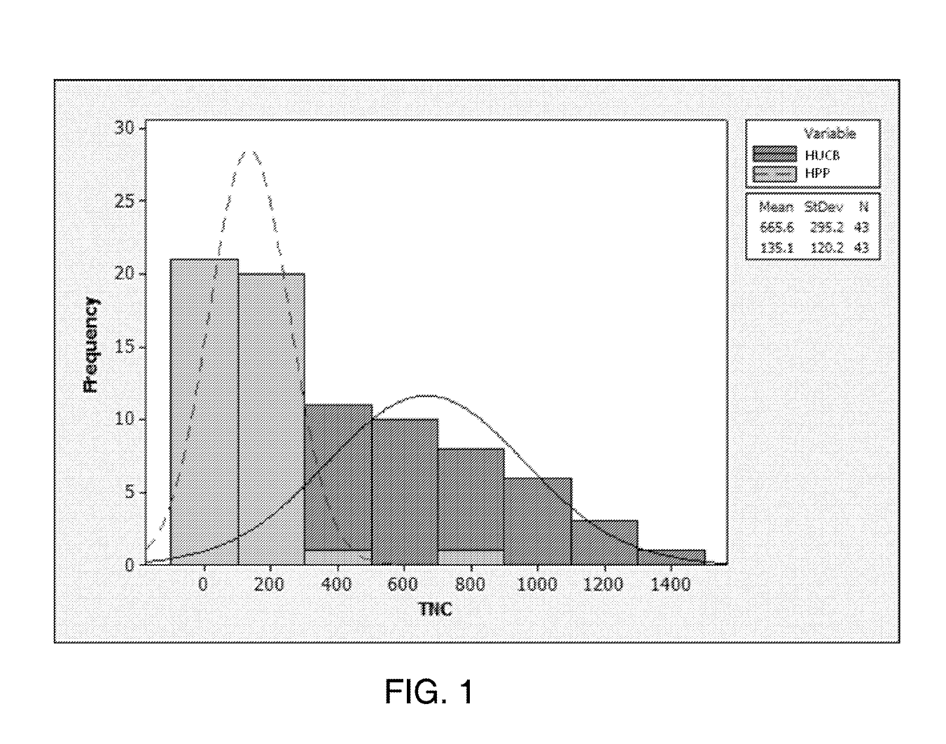

Forty-three pairs of donor-matched HPP and HUCB units were processed to determine total nucleated cell count. The average total nucleated cell count for a single unit of HPP was ˜135 million cells. The average total nucleated cell count for a single unit of HUCB was ˜666 million cells (FIG. 1).

### 6.3 Example 3: Progenitor Cell Populations in Human Placental Perfusate

This example illustrates the determination of the population of CD34⁺CD45⁻ and CD34⁺CD45⁺ cells in human placental perfusate and umbilical cord blood.

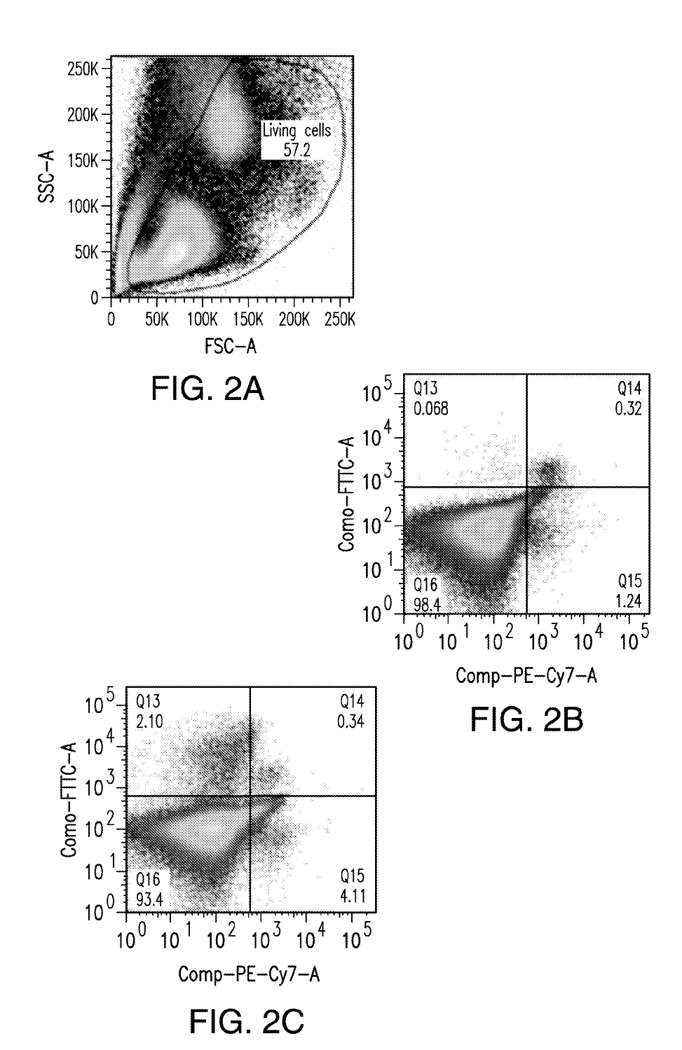

Fluorescence activated cell sorting (FACS) was used to determine subpopulations of human placental perfusate cells (FIG. 2A). A subpopulation of CD34⁺ cells are CD45⁻, therefore excluded for enumeration using ISHAGE protocol (Barnett, et al., 1999, Clin. Lab. Haem. 21:301-308), a sequential gating strategy (FIG. 2B), which gates for CD45⁺ cells first. A protocol using another sequential gating strategy was established whereby gating was done first for CD34⁺ cells, in order to analyze both CD34⁺CD45⁻ and CD34⁺CD45⁺ cells in human placental perfusate (FIG. 2C). Using this protocol, a distinct population of CD34⁺CD45⁻ cells was apparent in human placental perfusate.

Cell sorting by FACS was carried out as follows: Bags of donor matched HPP and HUCB were thawed at 370° C. separately, followed by RBC lysis by Ammonium chloride. The samples were then stained with FITC anti-human CD34 (Cat#555821, BD Biosciences) and PE anti-human CD45 (Cat#555483, BD Biosciences) for 15 min in the dark at RT. After two times wash with FACS buffer, the samples were resuspended at 1×10⁷ per ml and sorted by FACS Aria (BD Biosciences) using protocols provided by manufacturer.

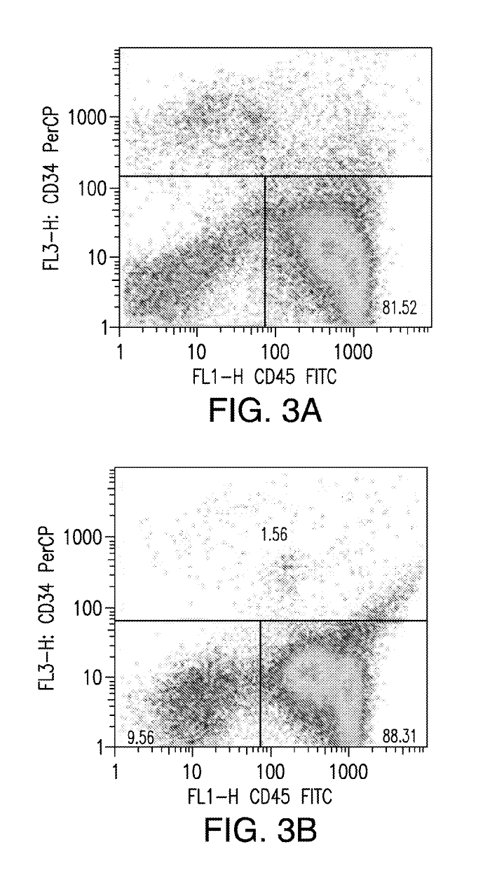

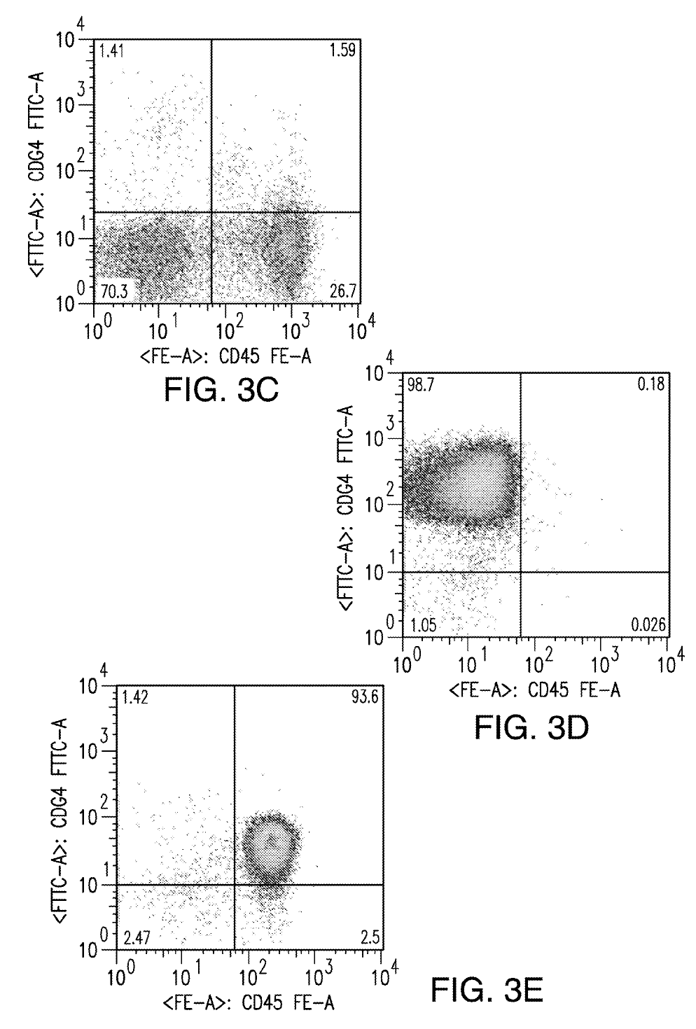

Using the FACS sorting protocol, it was determined that human placental perfusate contains a greater proportion of CD34⁺ cells compared to umbilical cord blood in donor-matched pairs (FIGS. 3A-3B). Colony-forming assays using human placental perfusate cells have demonstrated growth from CD34⁺CD45⁺ cells and CD34⁺CD45⁻ cells subsequent to sorting.

### 6.4 Example 4: CD34⁺ Subpopulations in Human Placental Perfusate

This example illustrates the determination of the population of CD34⁺CD31⁺, CD34⁺KDR⁺, and CD34⁺ CXCR-4⁺ cells in human placental perfusate and umbilical cord blood.

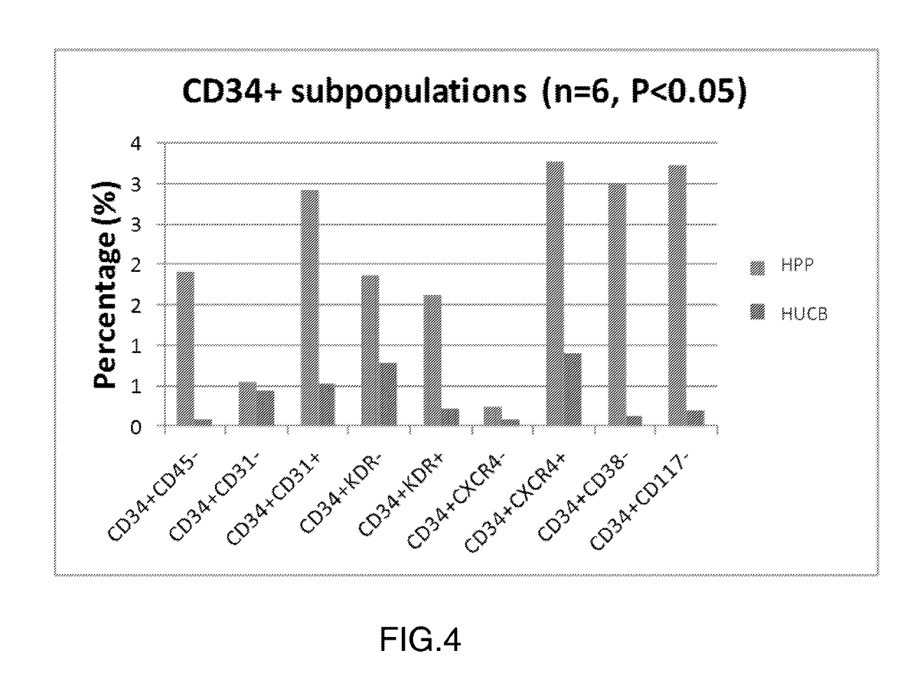

Using the phenotype characterization protocol as described in Section 6.1, it was determined that HPP CD34⁺ cells comprise a higher percentage of CD31⁺, KDR⁺, and CXCR-4⁺ cells than HUCB CD34⁺ cells (FIG. 4). These phenotypes are consistent with the HPP containing a population of hemangioblastic cells.

### 6.5 Example 5: Functional Evaluation of Cells from Human Placental Perfusate

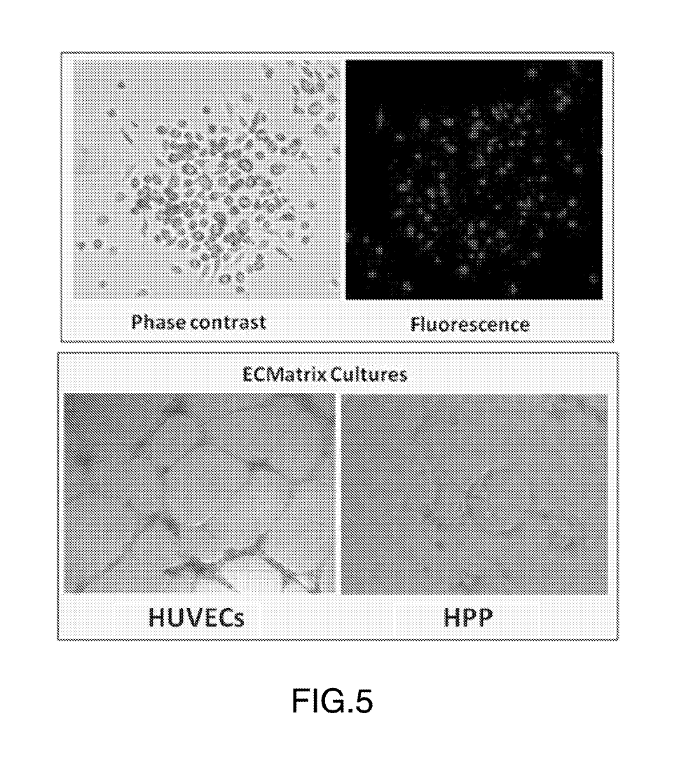

This example illustrates the determination of the angiogenic properties of human placental perfusate cells compared to umbilical cord blood cells. As demonstrated in FIG. 5, human placental perfusate showed higher angiogenesis (vessel-forming) activity compared to umbilical cord blood in the assay described herein.

HPP cells were obtained according to Section 5.5 above. HPP cells (FIG. 5, top left) were incubated with 10 μg/mL Dil-AC LDL(Cat#L3484, Life technology) at 37° C. for 4 h, fluorescence pictures of lipoprotein uptake by endothelial cells from HPP (FIG. 5, top right) were taken by Axiovert 200M (Zeiss). An in vitro functional assay was performed to assess the angiogenic properties of cells from human placental perfusate. HPP cells obtained according to Section 5.5 above, were cultured 18-24 hours on ECMATRIX™ at about 10⁶ cells per well in a 96-well plate using In Vitro Angiogenesis Assay Kit (Chemicon cat#ECM625), in which the cells are cultured in the presence of TGF-beta, FGF, plasminogen, tPA and matrix metalloproteases. Microvessel formation was observed in human placental perfusate cell culture (FIG. 5, bottom right). HUVECs (Human Umbilical Vein Endothelial Cells) were used as a positive control (FIG. 5, bottom left). No significant tube formation was observed in umbilical cord blood culture.

### 6.6 Example 6: Primitive Progenitor Cell Populations in Human Placental Perfusate

This example illustrates the determination of the populations of various CD34⁺ primitive progenitor cells in human placental perfusate and umbilical cord blood.

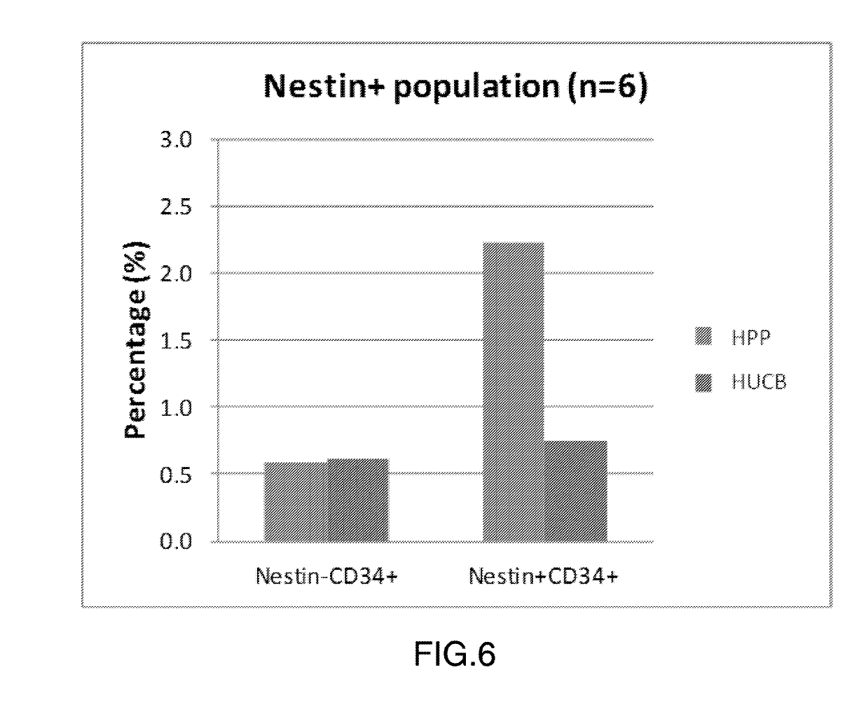

Using the phenotype characterization protocol as described in Section 6.1, it was determined that human placental perfusate contains a substantially larger proportion of Nestin⁺/CD34⁺ cells compared to umbilical cord blood (FIG. 6). Nestin⁺CD34⁺ cells are suggested to be more primitive neuronal progenitors (Mii et al., J. Cell Biol., 2013).

Human placental perfusate contains significantly larger quantities of immature hematopoietic stem cells populations (i.e., CD34⁺CD45⁻, CD34⁺CD38⁻) than umbilical cord blood, as shown in Table 5. Likewise shown in Table 2, the putatively hemangioblastic cell populations (i.e., CD34⁺C31⁺, CD34⁺KDR⁺, and CD34⁺ CXCR4⁺) are found in higher quantities in human placental perfusate than in umbilical cord blood.

TABLE 5
Primitive progenitors in human placental perfusate vs. umbilical cord blood.

| | HPP (n = 6) | | HUCB (n = 6) | |
|---|---|---|---|---|
| HSC Populations | Average | Range (min, max) | Average | Range (min, max) |
| CD34⁺ | 7.25 × 10⁶ | 6.62 × 10⁵, 2.44 × 10⁷ | 5.70 × 10⁶ | 7.20 × 10⁴, 1.87 × 10⁷ |
| CD34⁺CD45⁻ | 3.28 × 10⁶ | 5.00 × 10⁵, 1.00 × 10⁷ | 4.73 × 10⁵ | 2.00 × 10⁴, 1.68 × 10⁶ |
| CD34⁺CD31⁻ | 8.76 × 10⁵ | 2.06 × 10⁴, 2.45 × 10⁶ | 2.45 × 10⁶ | 1.40 × 10⁴, 8.02 × 10⁶ |
| CD34⁺CD31⁺ | 6.12 × 10⁶ | 6.30 × 10⁵, 2.35 × 10⁷ | 2.84 × 10⁶ | 4.88 × 10⁴, 9.70 × 10⁶ |
| CD34⁺KDR⁻ | 3.56 × 10⁶ | 1.62 × 10⁵, 9.62 × 10⁶ | 4.16 × 10⁶ | 1.63 × 10⁴, 1.68 × 10⁷ |
| CD34⁺KDR⁺ | 3.45 × 10⁶ | 5.11 × 10⁵, 1.49 × 10⁷ | 1.36 × 10⁶ | 4.56 × 10⁴, 5.72 × 10⁶ |
| CD34⁺CXCR4⁻ | 3.49 × 10⁵ | 6.72 × 10⁴, 7.14 × 10⁵ | 3.80 × 10⁵ | 4.67 × 10³, 1.78 × 10⁶ |
| CD34⁺CXCR4⁺ | 6.70 × 10⁶ | 5.62 × 10⁵, 2.38 × 10⁷ | 4.98 × 10⁶ | 6.08 × 10⁴, 1.62 × 10⁷ |
| CD34⁺CD38⁻ | 5.97 × 10⁶ | 6.49 × 10⁵, 2.26 × 10⁷ | 5.81 × 10⁵ | 5.20 × 10⁴, 2.45 × 10⁶ |
| CD34⁺CD117⁻ | 5.68 × 10⁶ | 7.56 × 10⁵, 1.91 × 10⁷ | 1.15 × 10⁶ | 5.44 × 10⁴, 2.02 × 10⁶ |
| CD34⁺CD140a⁺ | 4.39 × 10⁶ | 6.55 × 10⁵, 1.07 × 10⁷ | 7.99 × 10⁶ | 6.72 × 10⁴, 1.80 × 10⁷ |
| CD34⁺Nestin⁺ | 4.30 × 10⁶ | 3.67 × 10⁵, 1.25 × 10⁷ | 4.32 × 10⁶ | 2.40 × 10⁴, 1.10 × 10⁷ |

### 6.7 Example 7: T-Cell Content in Human Placental Perfusate

This example illustrates the determination of various T-cell populations in human placental perfusate and umbilical cord blood.

Overall class HLA I and II assessment, as well as extensive immmunophenotypic characterization was performed on human placental perfusate and umbilical cord blood using a 9-color T-cell FACS panel to depict T-cell subpopulations, CD45RA, CD8, CD25, CD127, CD69, CD3, CCR7, HLADR, and CD4, as in Section 6.1, above.

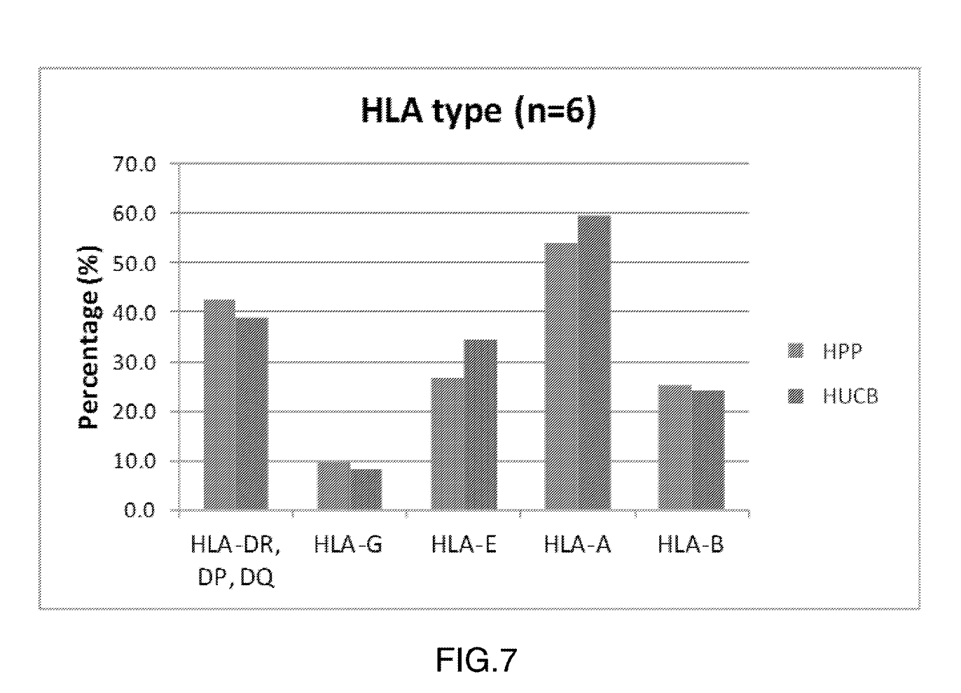

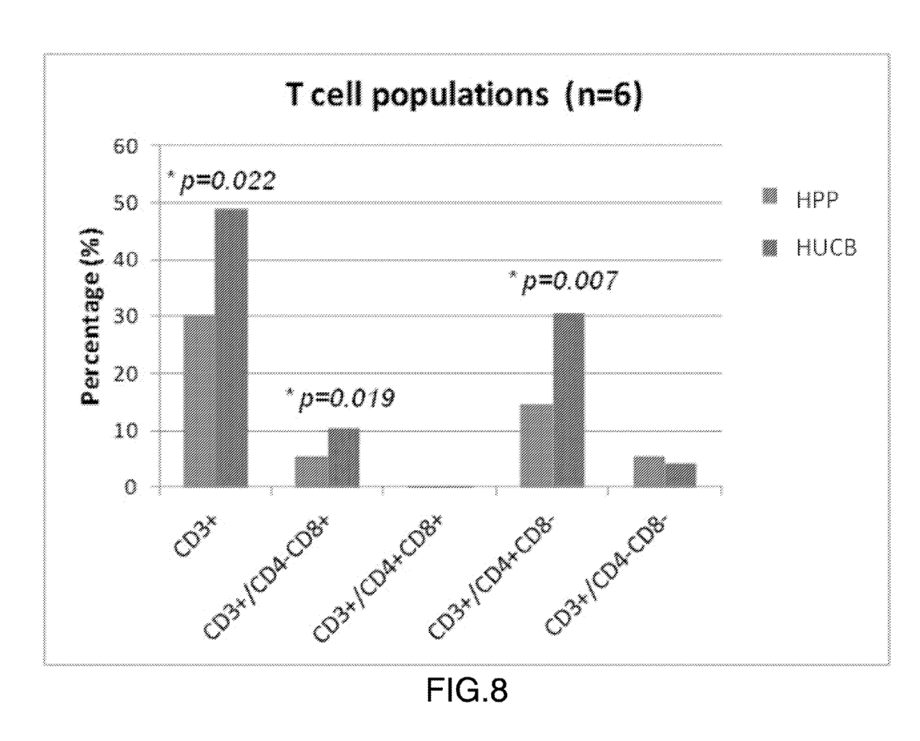

As shown in Table 6, the results demonstrate that human placental perfusate contains significantly lower T-cell content compared to umbilical cord blood. Likewise, human placental perfusate cells have a lower expression of HLA class I and HLA class II (FIG. 7). The relative proportions of specific T-cell populations expressing CD3, CD4, and/or CD8 were also determined in human placental perfusate and umbilical cord blood (FIG. 8). The T-cell content of human placental perfusate indicates, for example, the suitability of human placental perfusate cells for allogeneic-mismatched transplantation.

TABLE 6
T-cell populations in human placental perfusate and cord blood. “Hi” and
“low” indicate the expression intensity of a particular phenotypic marker.

| | | | | HUCB (n = 6) | |
|---|---|---|---|---|---|
| | | HPP (n = 6) | | | Range |
| T-cell Populations | | Average | Range (min, max) | Average | (min, max) |
| CD3⁺CD4⁺CD8⁻CD25hiCD127low | Treg | 1.88 × 10⁶ | 3.57 × 10⁴, 4.52 × 10⁶ | 8.57 × 10⁶ | 1.46 × 10⁶, |
| | | | | | 1.46 × 10⁷ |
| CD3⁺CD4⁺CD8⁻CD25hiCD127lowCD45RA⁺ | naive Treg | 6.37 × 10⁵ | 7.66 × 10³, 2.65 × 10⁵ | 3.09 × 10⁶ | 1.46 × 10⁶, |
| | | | | | 4.85 × 10⁶ |
| CD3⁺CD4⁺CD8⁻CD25hiCD127lowCD45RA⁻ | memory Treg | 1.40 × 10⁵ | 2.80 × 10⁴, 3.95 × 10⁵ | 1.29 × 10⁶ | 7.35 × 10³, |
| | | | | | 2.54 × 10⁶ |
| CD3⁺CD4⁺CD8⁻CD25hiCD127lowCD45RA⁻HLADR⁺ | HLADR⁺ memory Treg | 1.46 × 10⁴ | 1.47 × 10³, 3.43 × 10⁴ | 8.84 × 10⁴ | 7.62 × 10², |
| | | | | | 2.10 × 10⁵ |
| CD3⁺CD4⁺CD8⁻CD25^{+/-}CD127^{+/-} | CD4⁺ effector cells | 1.92 × 10⁷ | 1.12 × 10⁶, 4.22 × 10⁷ | 1.11 × 10⁸ | 1.88 × 10⁷, |
| | | | | | 1.65 × 10⁸ |
| CD3⁺CD4⁺CD8⁻CD25^{+/-}CD127^{+/-}CD45RA⁺HLADR⁻ | CD45RA⁺ (naive) CD4⁺ cells | 1.54 × 10⁷ | 4.67 × 10⁵, 3.49 × 10⁷ | 8.87 × 10⁷ | 1.85 × 10⁷, |
| | | | | | 1.44 × 10⁸ |
| CD3⁺CD4⁺CD8⁻CD25^{+/-}CD127^{+/-}CD45RA⁻CCR7⁺ | Central Memory CD4⁺ cells | 3.57 × 10⁶ | 6.43 × 10⁵, 1.16 × 10⁷ | 2.03 × 10⁷ | 7.73 × 10⁴, |
| | | | | | 3.21 × 10⁷ |
| CD3⁺CD4⁺CD8⁻CD25^{+/-}CD127^{+/-}CD45RA⁻CCR7⁻ | Effector Memory CD4⁺ cells | 3.35 × 10⁴ | 8.65 × 10³, 1.18 × 10⁵ | 7.19 × 10⁵ | 7.59 × 102, |
| | | | | | 2.29 × 10⁶ |
| CD3⁺CD4⁺CD8⁻CD25^{+/-}CD127^{+/-}CD45RA⁺CCR7⁻ | CD4⁺ terminal effector cells | 1.35 × 10⁵ | 5.62 × 10³, 5.49 × 10⁵ | 1.03 × 10⁶ | 2.62 × 10⁵, |
| | | | | | 2.81 × 10⁶ |
| CD3⁺CD4⁺CD8⁻CD25^{+/-}CD127^{+/-}CD45RA⁻HLADR⁺ | HLADR⁺ memory CD4⁺ cells | 3.66 × 10⁵ | 7.46 × 10⁴, 9.17 × 10⁵ | 1.82 × 10⁶ | 7.54 × 10⁴, |
| | | | | | 2.65 × 10⁶ |
| CD3⁺CD4⁺CD8⁻CD25^{+/-}CD127^{+/-}CD45RA⁻CD69⁺ | CD69⁺ memory CD4⁺ | 3.75 × 10⁴ | 3.93 × 10³, 8.86 × 10⁴ | 4.14 × 10⁵ | 5.65 × 10³, |
| | | | | | 7.85 × 10⁵ |
| CD3⁺CD4⁻CD8⁺ | CD8⁺ effector cells | 1.02 × 10⁷ | 2.40 × 10⁵, 2.35 × 10⁷ | 4.70 × 10⁷ | 9.44 × 10⁶, |
| | | | | | 8.91 × 10⁷ |
| CD3⁺CD4⁻CD8⁺CD45RA⁺HLADR⁻CCR7⁺ | naive CD8⁺ cells | 9.30 × 10⁶ | 2.52 × 10⁵, 2.26 × 10⁷ | 4.34 × 10⁷ | 9.07 × 10⁶, |
| | | | | | 8.38 × 10⁷ |
| CD3⁺CD4⁻CD8⁺CD45RA⁺CCR7⁺ | CD8⁺ central memory | 7.41 × 10⁵ | 8.40 × 10⁴, 1.79 × 10⁶ | 3.14 × 10⁶ | 1.98 × 10⁴, |
| | | | | | 5.88 × 10⁶ |
| CD3⁺CD4⁻CD8⁺CD45RA⁺CCR7⁻ | CD8⁺ terminal effector cells | 1.13 × 10⁵ | 3.03 × 10³, 5.36 × 10⁵ | 3.10 × 10⁵ | 1.22 × 10⁵, |
| | | | | | 5.44 × 10⁵ |
| CD3⁺CD4⁻CD8⁺CD45RA⁻CCR7⁻ | CD8⁺effector memory cells | 1.75 × 10⁴ | 9.18 × 10², 5.92 × 10⁴ | 8.53 × 10⁴ | 2.08 × 10³, |
| | | | | | 1.82 × 10⁵ |
| CD3⁺CD4⁻CD8⁺CD45RA⁻HLADR⁺ | HLADR⁺ memory CD8⁺ cells | 3.73 × 10⁴ | 5.10 × 10², 9.12 × 10⁴ | 2.22 × 10⁵ | 1.13 × 10³, |
| | | | | | 6.57 × 10⁵ |
| CD3⁺CD4⁺CD8⁺ | CD4⁺CD8⁺ lymphocytes. | 1.68 × 10⁵ | 1.71 × 10⁴, 6.16 × 10⁵ | 5.96 × 10⁵ | 2.31 × 10⁵, |
| | | | | | 1.26 × 10⁶ |
| CD3⁺CD4⁻CD8⁻ | CD4⁻CD8⁻ lymphocytes | 9.57 × 10⁶ | 7.93 × 10⁵, 2.34 × 10⁷ | 2.16 × 10⁷ | 3.50 × 10⁶, |
| | | | | | 5.47 × 10⁷ |
| CD3⁺CD4⁻CD8⁻CD69⁺ | CD69⁺ double negative | 2.67 × 10⁵ | 1.06 × 10⁴, 5.01 × 10⁵ | 1.26 × 10⁶ | 1.98 × 10⁵, |
| | lymphocytes | | | | 4.02 × 10⁶ |

### 6.8 Example 8: T-Cell Isolation, Functional Evaluation, and Expansion

This example illustrates methods that can be used to successfully isolate, evaluate, and expand populations of T_{reg} cells in human placental perfusate and umbilical cord blood. Similar methods may be used to isolate, evaluate, and expand other populations or subpopulations of human placental perfusate cells.

A complete kit for human CD4⁺CD127^{low} CD25⁺ regulatory T cells (Cat#15861, StemCell) can be used for isolation T_{reg} cells from donor matched HPP or HUCB separately. Isolated T_{reg} cells from donor matched HPP or HUCB separately, donor matched HPP or HUCB, or donor matched HPP or HUCB without T_{reg} cells can be evaluated by an in vitro Bead T-cell Reaction (BTR) assay. In brief, T cells from peripheral blood (PB) activated with anti-CD3/CD28 beads can be cocultured with the samples listed above for 5 days. The suppression of proliferation of CD4 and CD8 T cells can be measured by FACS.

Two beads based expansion kit can be evaluated for T_{reg} cell expansion from donor matched HPP and HUCB separately using a T_{reg} expansion kit (Cat#: 130-095-345, Miltenyi) and a DYNABEADS® Regulatory CD4⁺CD25⁺ T Cell Kit (Cat#11363D, Life Technology). Improvement of the potency of expanded Treg cells for clinical use may be accomplished using necrosis factor receptor family members: OX40, 4-1BB for enhancement (Hippen et al, 2008)

### EQUIVALENTS

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed:

1. A composition comprising isolated human placental perfusate, wherein the human placental perfusate comprises at least 6×10⁵ CD34⁺ cells.

2. The composition of claim 1, wherein the composition further comprises a 2-fold greater number of CD34⁺ cells.

3. The composition of claim 1, wherein the composition further comprises a 10-fold greater number of CD34⁺ cells.

4. The composition of claim 1, wherein the composition further comprises a 50-fold greater number of CD34⁺ cells.

5. The composition of claim 1, wherein the composition comprises substantially pure human placental perfusate CD34⁺ cells.

6. A composition comprising isolated human placental perfusate, wherein the human placental perfusate comprises at least 5×10⁵ CD34⁺CD45⁻ cells.

7. The composition of claim 6, wherein the composition further comprises a 2-fold greater number of CD34⁺CD45⁻ cells.

8. The composition of claim 6, wherein the composition further comprises a 10-fold greater number of CD34⁺CD45⁻ cells.

9. The composition of claim 6, wherein the composition further comprises a 50-fold greater number of CD34⁺CD45⁻ cells.

10. The composition of claim 6, wherein the composition comprises substantially pure human placental perfusate CD34⁺CD45⁻ cells.

11. A composition comprising isolated human placental perfusate, wherein the human placental perfusate comprises at least 6×10⁵ CD34⁺CD31⁺ cells.

12. The composition of claim 11, wherein the composition further comprises a 2-fold greater number of CD34⁺CD31⁺ cells.

13. The composition of claim 11, wherein the composition further comprises a 10-fold greater number of CD34⁺CD31⁺ cells.

14. The composition of claim 11, wherein the composition further comprises a 50-fold greater number of CD34⁺CD31⁺ cells.

15. The composition of claim 11, wherein the composition comprises substantially pure human placental perfusate CD34⁺CD31⁺ cells.

16. A composition comprising isolated human placental perfusate, wherein the human placental perfusate comprises at least 5×10⁵CD34⁺KDR⁺ cells.

17. The composition of claim 16, wherein the composition further comprises a 2-fold greater number of CD34⁺KDR⁺ cells.

18. The composition of claim 16, wherein the composition further comprises a 10-fold greater number of CD34⁺KDR⁺ cells.

19. The composition of claim 16, wherein the composition further comprises a 50-fold greater number of CD34⁺KDR⁺ cells.

20. The composition of claim 16, wherein the composition comprises substantially pure human placental perfusate CD34⁺KDR⁺ cells.

21. A composition comprising isolated human placental perfusate, wherein the human placental perfusate comprises at least 5×10⁵CD34⁺ CXCR4⁺ cells.

22. The composition of claim 21, wherein the composition further comprises a 2-fold greater number of CD34⁺ CXCR4⁺ cells.

23. The composition of claim 21, wherein the composition further comprises a 10-fold greater number of CD34⁺ CXCR4⁺ cells.

24. The composition of claim 21, wherein the composition further comprises a 50-fold greater number of CD34⁺CXCR4⁺ cells.

25. The composition of claim 21, wherein the composition comprises substantially pure human placental perfusate CD34⁺CXCR4⁺ cells.

26. A composition comprising isolated human placental perfusate, wherein the human placental perfusate comprises at least 6×10⁵CD34⁺CD38⁻ cells.

27. The composition of claim 26, wherein the composition further comprises a 2-fold greater number of CD34⁺CD38⁻ cells.

28. The composition of claim 26, wherein the composition further comprises a 10-fold greater number of CD34⁺CD38⁻ cells.

29. The composition of claim 26, wherein the composition further comprises a 50-fold greater number of CD34⁺CD38⁻ cells.

30. The composition of claim 26, wherein the composition comprises substantially pure human placental perfusate CD34⁺CD38⁻ cells.

31. A composition comprising isolated human placental perfusate, wherein the human placental perfusate comprises at least 7×10⁵CD34⁺CD117⁻ cells.

32. The composition of claim 31, wherein the composition further comprises a 2-fold greater number of CD34⁺CD117⁻ cells.

33. The composition of claim 31, wherein the composition further comprises a 10-fold greater number of CD34⁺CD117⁻ cells.

34. The composition of claim 31, wherein the composition further comprises a 50-fold greater number of CD34⁺CD117⁻ cells.

35. The composition of claim 31, wherein the composition comprises substantially pure human placental perfusate CD34⁺CD117⁻ cells.

36. A composition comprising isolated human placental perfusate, wherein the human placental perfusate comprises at least 6×10⁵CD34⁺CD140a⁺ cells.

37. The composition of claim 36, wherein the composition further comprises a 2-fold greater number of CD34⁺CD140a⁺ cells.

38. The composition of claim 36, wherein the composition further comprises a 10-fold greater number of CD34⁺CD140a⁺ cells.

39. The composition of claim 36, wherein the composition further comprises a 50-fold greater number of CD34⁺CD140a⁺ cells.

40. The composition of claim 36, wherein the composition comprises substantially pure human placental perfusate CD34⁺CD140a⁺ cells.

41. A composition comprising isolated human placental perfusate, wherein the human placental perfusate comprises at least 3×10⁵CD34⁺Nestin⁺ cells.

42. The composition of claim 40, wherein the composition further comprises a 2-fold greater number of CD34⁺Nestin⁺ cells.

43. The composition of claim 40, wherein the composition further comprises a 10-fold greater number of CD34⁺Nestin⁺ cells.

44. The composition of claim 40, wherein the composition further comprises a 50-fold greater number of CD34⁺Nestin⁺ cells.

45. The composition of claim 40, wherein the composition is substantially pure human placental perfusate CD34⁺Nestin⁺ cells.

46. A composition comprising isolated human placental perfusate, wherein the human placental perfusate comprises at least 3×10⁴CD3⁺CD4⁺CD8⁻CD25^{hi}CD127^{low} cells.

47. The composition of claim 46, wherein the composition further comprises a 2-fold greater number of CD3⁺CD4⁺CD8⁻CD25^{hi}CD127^{low} cells.

48. The composition of claim 46, wherein the composition further comprises a 10-fold greater number of CD3⁺CD4⁺CD8⁻CD25^{hi}CD127^{low} cells.

49. The composition of claim 46, wherein the composition further comprises a 50-fold greater number of CD3⁺CD4⁺CD8⁻CD25^{hi}CD127^{low} cells.

50. The composition of claim 46, wherein the composition is substantially pure human placental perfusate CD3⁺CD4⁺CD8⁻CD25^{hi}CD127^{low} cells.

51. The composition of any one of claims 1 to 50, wherein the human placental perfusate has been isolated from perfusion of a single placenta.

52. A method of treating a central nervous system injury, disease, or disorder in a subject, comprising administering to the subject the composition of any one of claims 1 to 50 and hematopoietic cells from another source.

53. The method of claim 52, wherein said central nervous system injury, disease, or disorder is hypoxic ischemic encephalopathy.

54. A method of treating sarcopenia in a subject, comprising administering to the subject the composition of any one of claims 1 to 50 and hematopoietic cells from another source.

55. A method of inducing chimerism in a subject, comprising administering to the subject the composition of any one of claims 1 to 50 and hematopoietic cells from another source.

56. A method for cell engraftment in a subject, comprising administering to the subject the composition of any one of claims 1 to 50 and hematopoietic cells from another source.

57. A method for reducing the duration or severity of graft versus host disease (GVHD) in a subject, comprising administering to the subject the composition of any one of claims 1 to 50 and hematopoietic cells from another source.

58. A method of treating a metabolic disorder in a subject, comprising administering to the subject the composition of any one of claims 1 to 50 and hematopoietic cells from another source.

59. A method of treating a hematologic disorder or malignancy in a subject, comprising administering to the subject the composition of any one of claims 1 to 50 and hematopoietic cells from another source.

60. A composition as defined in any one of claims 1 to 50 for use in a method:
(a) of treatment of a central nervous system injury, disease, or disorder in a subject, preferably said central nervous system injury, disease, or disorder is hypoxic ischemic encephalopathy;
(b) of inducing chimerism in a subject;
(c) for cell engraftment;
(d) for reducing the duration or severity of graft versus host disease (GVHD) in a subject;
(e) of treating a metabolic disorder in a subject;
(f) of treating a hematologic disorder or malignancy in a subject; or
(g) of treating sarcopenia in a subject.

61. The composition for use of claim 60, wherein the composition further comprises hematopoietic cells from another source.
